(19) United States
(12) Patent Application Publication
(10) Pub. No.: US 2025/0241802 A1
(43) Pub. Date: Jul. 31, 2025

(54) DUAL ZONED WEB MATERIAL

(71) Applicant: Kimberly-Clark Worldwide, Inc., Neenah, WI (US)

(72) Inventors: Marta Embid, Reigate (GB); Ilona Herzigová, Malé Svatonovice (CZ); Vladimír Dvorácek, Dvur Králové nad Labem (CZ); Alexander Schmidt-Forst, Erlangen (DE); Dmitry Vnukov, Stupino (RU); Cory M. Koplien, Weyauwega, WI (US)

(21) Appl. No.: 18/855,006

(22) PCT Filed: Apr. 3, 2023
(86) PCT No.: PCT/US23/17291

§ 371 Date: Oct. 8, 2024

Related U.S. Application Data
(60) Provisional application No. 63/329,014, filed on Apr. 8, 2022.

(51) Int. Cl. A61F 13/511; A61F 13/496; A61F 13/512; A61F 13/513

(52) U.S. Cl. CPC A61F 13/51104; A61F 13/496; A61F 13/512; A61F 13/513

(57) ABSTRACT

Absorbent articles and web materials are disclosed. In one embodiment, an absorbent article comprises a bodyside liner; a backsheet; an absorbent body; and a topical liner material coupled to the bodyside liner material, the topical liner material comprises a first and a second zone, the first and second zones are individually contiguous and visually distinct, the first zone comprises embossment, aperture, and protrusion morphological features, the embossment features extending from the first body facing surface toward the second garment facing surface and the protrusion features protruding from the second garment facing surface and toward the backsheet material, wherein the second zone comprises protrusion and aperture morphological features, and the features of each of the first and second zones define outer boundaries, the outer boundaries of the zones forming a first shape and second shape different from the first shape.

### TECHNICAL FIELD

The present disclosure relates web materials with at least two distinct zones. More particularly, the present disclosure relates to web materials with at least two distinct zones configured to efficiently handle different bodily exudates and absorbent articles including such web materials.

### BACKGROUND OF THE DISCLOSURE

A primary function of personal care absorbent articles is to absorb and retain bodily exudates such as urine, fecal material, blood, and menses with additional desired attributes including low leakage of the exudates from the absorbent article and a dry feel to the wearer of the absorbent article. By preventing leakage of the exudates from the absorbent article, the absorbent article intends to prevent the bodily exudates from soiling or contaminating a wearer's or caregiver's clothing or other articles, such as bedding, that can come in contact with the wearer.

Bodily exudates come in different forms and qualities. For example, urine exudates and bowel movement (BM) exudates have differing properties which affect how they flow and their uptake by absorbent articles. As well, such bodily exudates have a different effect on a wearer's skin, and such effects may occur after different lengths of exposure to the wearer's skin. Accordingly, there is a need for materials and absorbent articles that effectively and efficiently handle such different bodily exudates to help ensure sustained skin health of a wearer of such absorbent articles.

### SUMMARY OF THE DISCLOSURE

In a first embodiment, an absorbent article extending in a longitudinal direction and a transverse direction may comprise: a bodyside liner material having a body facing surface and a garment facing surface; a backsheet material; an absorbent body disposed between the bodyside liner material and the backsheet material; and a topical liner material coupled to the body facing surface of the bodyside liner material and having a depth dimension extending between a first body facing surface and a second, opposed garment facing surface, the topical liner material comprising a first zone and a second zone separated in the longitudinal direction, the first zone and the second zone each being individually contiguous and visually distinct, wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more protrusion and one or more aperture morphological features, and wherein an average area of the aperture morphological features of the first zone are larger than an average area of the aperture morphological features of the second zone, and wherein: the protrusion morphological features of the first zone have proximal ends and distal ends, the embossment morphological features of the first zone have an opening comprising an opening width, a portion having an interior minimum width, and a portion having an interior maximum width with the portion having the interior minimum width disposed between, in a depth direction of the topical liner material, the opening and the portion having the interior maximum width, and the protrusion morphological features are disposed within the embossment morphological features such that the proximal ends of the protrusion morphological features are disposed between, in a depth direction of the topical liner material, the portion of the embossment having the interior maximum width and the portion of the embossment having the interior minimum width.

In another embodiment, an absorbent article extending in a longitudinal direction and a transverse direction may comprise: a bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct; a backsheet material; and an absorbent body disposed between the bodyside liner material and the backsheet material; wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more protrusion morphological features, and wherein: the protrusion morphological features of the first zone have proximal ends and distal ends, the embossment morphological features of the first zone each have an opening comprising an opening width, interior portions defined by sidewalls of the embossment morphological features having an interior minimum width and an interior maximum width, with the interior minimum width disposed between the opening and the interior maximum width, and the protrusion morphological features are disposed within the embossment morphological features such that the proximal ends of the protrusion morphological features are disposed between, in the depth direction of the bodyside liner material, the portions of the embossment morphological features which have the interior maximum width and the interior minimum width.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is side perspective view of an exemplary embodiment of an absorbent article, such as a diaper, in a fastened condition.

FIG. 2 is a top plan view of the absorbent article of FIG. 1 in a stretched, laid flat, unfastened condition, including a dual zoned web according to aspects of the present disclosure.

FIG. 3 is a front perspective view of an alternative embodiment of an absorbent article, such as a pant.

FIG. 4 is a top plan view of the absorbent article of FIG. 3 in a stretched, laid flat and open condition and including a dual zoned web according to aspects of the present disclosure.

FIG. 5 is a schematic depiction of an exemplary web material having first and second visually distinct zones according to aspects of the present disclosure.

FIGS. 6A-6E are schematics of exemplary shapes that may be formed by zones of web materials according to aspects of the present disclosure.

FIG. 7 is an optical image of an exemplary web according to aspects of the present disclosure comprising first and second visually distinct zones with outer boundaries of the first and second zones highlighted.

FIG. 8 is a close-up of the first zone of the optical image of FIG. 7.

FIG. 9 is an optical image captured by a stereo microscope of morphological features of the first zone of FIG. 7.

FIG. 10 is an optical image captured by a stereo microscope of a cross-section of a morphological feature of the first zone of FIG. 7.

FIG. 11 is a close-up of the second zone of the optical image of FIG. 7.

FIG. 12 is a schematic depiction of an exemplary absorbent article chassis comprising an exemplary web material having first and second zones according to aspects of the present disclosure.

FIG. 13 is an optical image of an alternative exemplary web according to aspects of the present disclosure comprising first and second visually distinct zones with outer boundaries of the first and second zones highlighted.

FIG. 14 is a close-up of the first zone of the optical image of FIG. 13.

Repeat use of reference characters in the present specification and drawings is intended to represent the same or analogous features or elements of the disclosure.

### DETAILED DESCRIPTION OF THE DISLOSURE

In an embodiment, the present disclosure is generally directed towards a web material comprising at least two visually and functionally distinct zones. The two visually and functionally distinct zones are generally differently adapted to handle urine exudates and BM exudates. Such dual zoned web materials may be used within an absorbent article to handle such urine and BM exudates effectively and efficiently, in terms of helping with their uptake into the absorbent article and away from a wearer's skin and limiting their spread to help prevent wide-spread initial contact with a wearer's skin. Each example described herein is provided by way of explanation and is not meant as a limitation. For example, features illustrated or described as part of one embodiment/example or figure can be used on another embodiment/example or figure to yield yet another embodiment/example. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “acquisition layer” refers herein to a layer capable of accepting and temporarily holding liquid bodily exudates to decelerate and diffuse a surge or gush of the liquid bodily exudates and to subsequently release the liquid bodily exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid bodily exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article

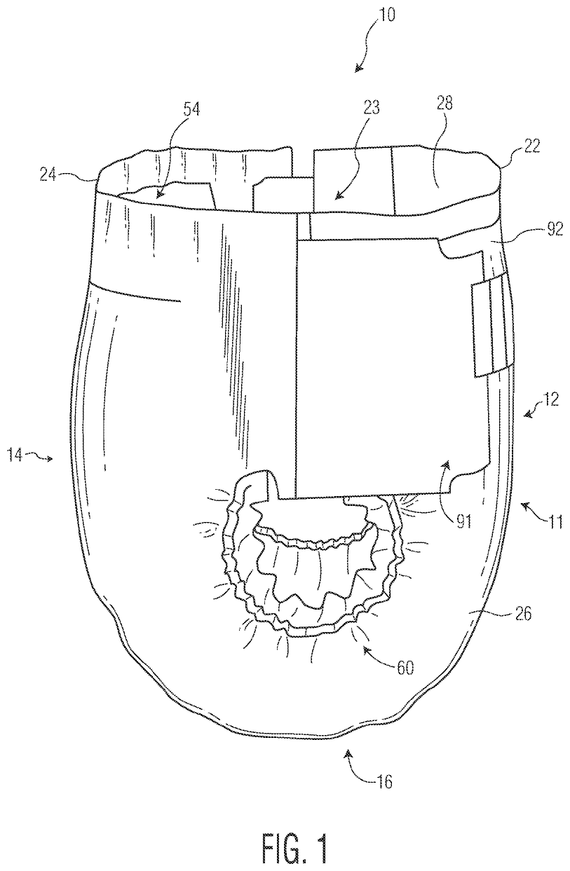

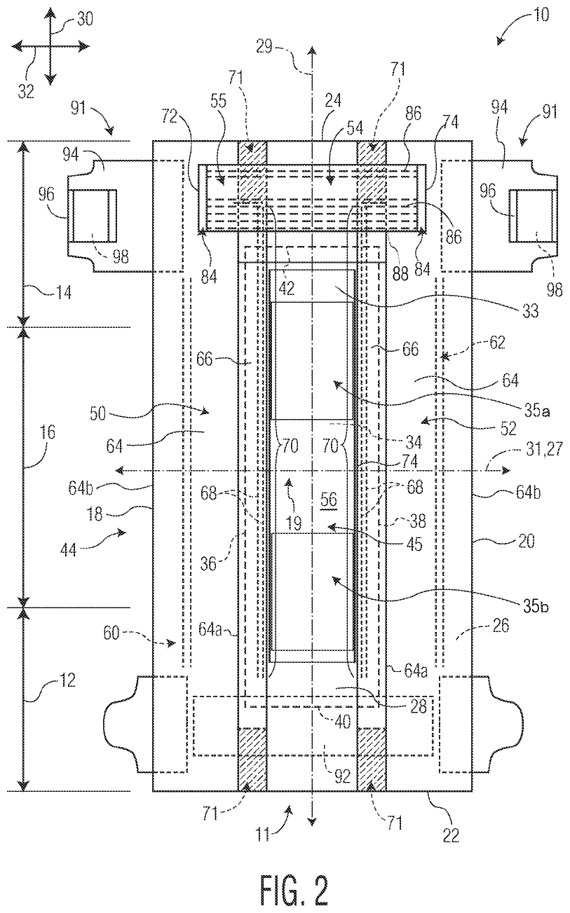

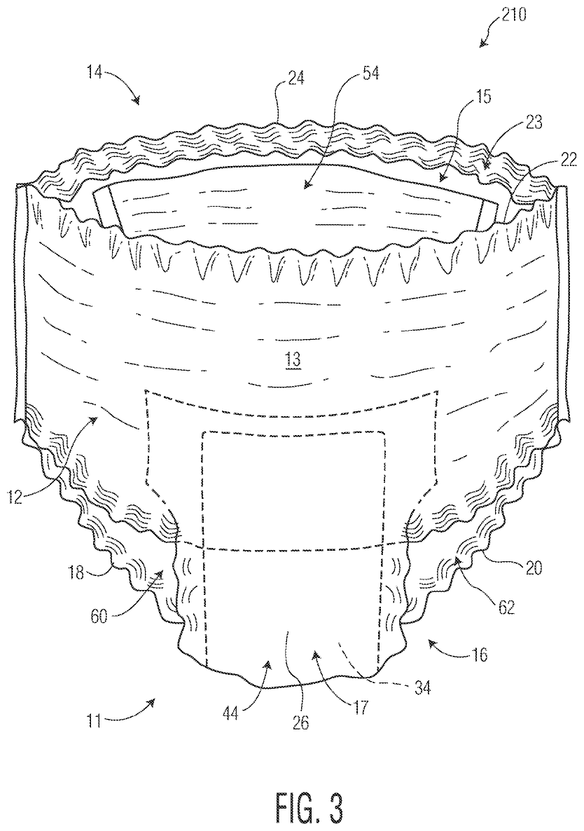

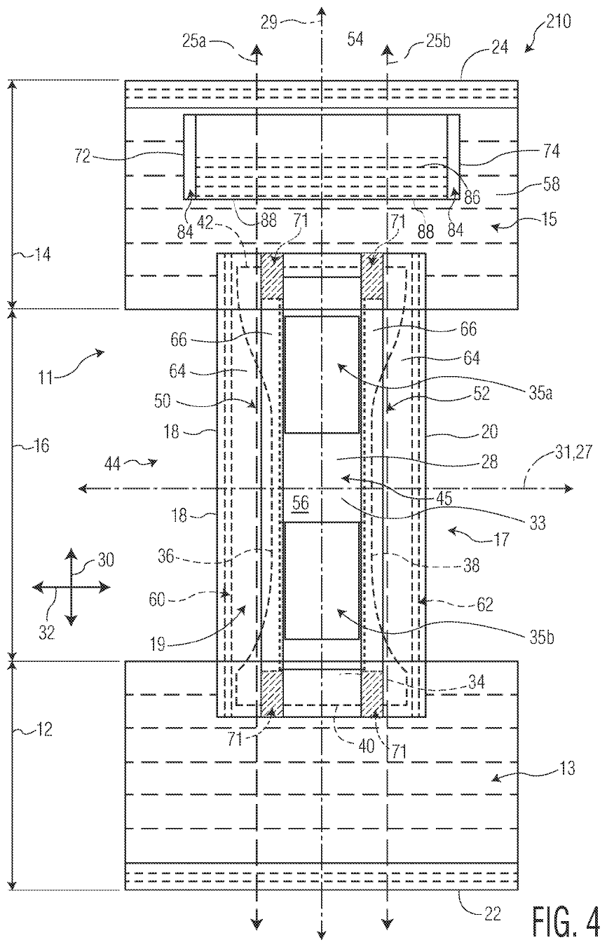

Referring to FIGS. 1-2, a non-limiting illustration of an absorbent article 10, for example a diaper, is illustrated. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross-machine direction manufacturing of a product, without departing from the spirit and scope of the disclosure. For example, the absorbent article 210 in FIGS. 3-4 provide an exemplary embodiment of an absorbent article 210 that can be manufactured in cross-machine direction manufacturing process.

The absorbent article 10 illustrated in FIGS. 1 and 2 and the absorbent article 210 illustrated in FIGS. 3 and 4 can each include a chassis 11. The absorbent article 10, 210 can include a front waist region 12, a rear waist region 14, and a crotch region 16 disposed between the front waist region 12 and the rear waist region 14 and interconnecting the front and rear waist regions, 12, 14, respectively.

The front waist region 12 can be referred to as the front-end region, the rear waist region 14 can be referred to as the rear-end region, and the crotch region 16 can be referred to as the intermediate region. In the embodiment depicted in FIGS. 3 and 4, a three-piece construction of an absorbent article 210 is depicted where the absorbent article 210 can have a chassis 11 including a front waist panel 13 defining the front waist region 12, a rear waist panel 15 defining the rear waist region 14, and an absorbent panel 17 defining the crotch region 16 of the absorbent article 210. The absorbent panel 17 can extend between the front waist panel 13 and the rear waist panel 15. In some embodiments, the absorbent panel 17 can overlap the front waist panel 13 and the rear waist panel 15. The absorbent panel 17 can be bonded to the front waist panel 13 and the rear waist panel 15 to define a three-piece construction. However, it is contemplated that an absorbent article can be manufactured in a cross-machine direction without being a three-piece construction garment.

The absorbent article 10, 210 can have a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIGS. 1 and 2. In other embodiments, the longitudinal side edges 18, 20 can be curved between the front waist edge 22 and the rear waist edge 24. In the absorbent article 210 of FIGS. 3 and 4, the longitudinal side edges 18, 20 can include portions of the front waist panel 13, the absorbent panel 17, and the rear waist panel 15.

The front waist region 12 can include the portion of the absorbent article 10, 210 that, when worn, is positioned at least in part on the front of the wearer while the rear waist region 14 can include the portion of the absorbent article 10, 210 that, when worn, is positioned at least in part on the back of the wearer. The crotch region 16 of the absorbent article 10, 210 can include the portion of the absorbent article 10, 210 that, when worn, is positioned between the legs of the wearer and can partially cover the lower torso of the wearer. The waist edges, 22 and 24, of the absorbent article 10, 210 are configured to encircle the waist of the wearer and together define a central waist opening 23 (as labeled in FIG. 1 and FIG. 3) for the waist of the wearer. Portions of the longitudinal side edges 18, 20 in the crotch region 16 can generally define leg openings for the legs of the wearer when the absorbent article 10, 210 is worn.

The absorbent article 10, 210 can include an outer cover 26 and a bodyside liner 28. The outer cover 26 and the bodyside liner 28 can form a portion of the chassis 11. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIGS. 2 and 4, the absorbent article 10, 210 can have a longitudinal axis 29 extending in the longitudinal direction 30 and a lateral axis 31 extending in the lateral direction 32.

The chassis 11 can include an absorbent body 34. The absorbent body 34 can be disposed between the outer cover 26 and the bodyside liner 28. The absorbent body 34 can have longitudinal edges, 36 and 38, which, in an embodiment, can form portions of the longitudinal side edges, 18 and 20, respectively, of the absorbent article 10, 210. The absorbent body 34 can have a first end edge 40 that is opposite a second end edge 42, respectively, which, in an embodiment, can form portions of the waist edges, 22 and 24, respectively, of the absorbent article 10. In some embodiments, the first end edge 40 can be in the front waist region 12. In some embodiments, the second end edge 42 can be in the rear waist region 14. In an embodiment, the absorbent body 34 can have a length and width that are the same as or less than the length and width of the absorbent article 10, 210. The bodyside liner 28, the outer cover 26, and the absorbent body 34 can form part of an absorbent assembly 44. In the absorbent article 210 of FIGS. 3 and 4, the absorbent panel 17 can form the absorbent assembly 44. The absorbent assembly 44 can also include a fluid transfer layer (not shown) and/or a fluid acquisition layer (not shown) between the bodyside liner 28 and the fluid transfer layer as is known in the art. The absorbent assembly 44 can also include a spacer layer (not shown) disposed between the absorbent body 34 and the outer cover 26.

The absorbent article 10, 210 can be configured to contain and/or absorb liquid, solid, and semi-solid bodily exudates discharged from the wearer. In some embodiments, containment flaps 50, 52 can be configured to provide a barrier to the lateral flow of bodily exudates. To further enhance containment and/or absorption of bodily exudates, the absorbent article 10, 210 can suitably include a waist containment member 54. In some embodiments, the waist containment member 54 can be disposed in the rear waist region 14 of the absorbent article 10, 210. Although not depicted herein, it is contemplated that the waist containment member 54 can be additionally or alternatively disposed in the front waist region 12 of the absorbent article 10, 210.

The waist containment member 54 can be disposed on the body facing surface 19 of the chassis 11 to help contain and/or absorb bodily exudates. In some embodiments, such as in the absorbent articles 10 depicted in FIGS. 1 and 2, the waist containment member 54 can be disposed on the body facing surface 45 of the absorbent assembly 44. In some embodiments, the waist containment member 54 can be disposed on the body facing surface 56 of the bodyside liner 28. In some embodiments, such as in the absorbent article 210 depicted in FIGS. 3 and 4, the waist containment member 54 can be disposed on the body facing surface 58 of the rear waist panel 15.

The absorbent article 10, 210 can further include leg elastic members 60, 62 as are known to those skilled in the art. The leg elastic members 60, 62 can be attached to the outer cover 26 and/or the bodyside liner 28 along the opposite longitudinal side edges, 18 and 20, and positioned in the crotch region 16 of the absorbent article 10, 210. The leg elastic members 60, 62 can be parallel to the longitudinal axis 29 as shown in FIGS. 2 and 4 or can be curved as is known in the art. The leg elastic members 60, 62 can be elastomeric and can provide elasticized leg cuffs.

In some embodiments, the absorbent article 10, 210 can further include longitudinal extending fold lines 25a, 25b, as shown in FIGS. 2 and 4. The first longitudinal extending fold line 25a can be on one side of the longitudinal axis 29 of the absorbent article 10, 210 and the second longitudinal extending fold line 25b can be on an opposite side of the longitudinal axis 29. In some embodiments, the longitudinal extending fold lines 25a, 25b can be generally parallel to the longitudinal axis 29 of the absorbent article 10, 210. In some embodiments, the absorbent article 10, 210 can further include a lateral extending fold line 27. The lateral extending fold line 27 can be parallel to and located at the lateral axis 31 of the absorbent article 10, 210 in some embodiments.

Additional details regarding each of these elements of the absorbent article 10, 210 described herein can be found below and with reference to the Figures.

### Outer Cover

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10, 210. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two-layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid bodily exudates from leaking out of the absorbent article 10, 210 and wetting articles, such as bed sheets and clothing, as well as the wearer and caregiver.

In some embodiments, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

### Bodyside Liner

The bodyside liner 28 of the absorbent article 10, 210 can overlay the absorbent body 34 and the outer cover 26 and can isolate the wearer's skin from liquid waste retained by the absorbent body 34. In various embodiments, a fluid transfer layer can be positioned between the bodyside liner 28 and the absorbent body 34. In various embodiments, an acquisition layer (not shown) can be positioned between the bodyside liner 28 and the absorbent body 34 or a fluid transfer layer, if present. In various embodiments, the bodyside liner 28 can be bonded to the acquisition layer, or to the fluid transfer layer if no acquisition layer is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 34 and/or a fluid transfer layer, if present, and/or an acquisition layer, if present, and/or a spacer layer, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. The bodyside liner 28 may be narrower than the outer cover 26. However, in other embodiments, the bodyside liner 28 and the outer cover 26 may be of the same dimensions in width and length. In other embodiments, the bodyside liner 28 can be of greater width than the outer cover 26. It is also contemplated that the bodyside liner 28 may not extend beyond the absorbent body 34 and/or may not be secured to the outer cover 26. In some embodiments, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit bodily exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,474,660 to Kirby, Scott S. C. et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from 10 or 12 to 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10, 210. In other aspects, the bodyside liner 28 can be stretchable, and more suitably elastic, in both the lateral and the longitudinal directions 32, 30, respectively.

### Absorbent Body

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10, 210. The absorbent body 34 can have a length and width that can be less than or equal to the length and width of the absorbent article 10, 210.

In an embodiment, the absorbent body 34 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 34 can be a matrix of cellulosic fluff and superabsorbent material. In further embodiments, the absorbent body 34 can comprise mostly superabsorbent material, or even greater than 80% superabsorbent material, greater than 90% superabsorbent material, or comprise 100% superabsorbent material, by weight of absorbent material of the absorbent body 34. Although, in other embodiments, the absorbent body 34 can be free of superabsorbent material. In an embodiment, the absorbent body 34 may be constructed of a single layer of materials, or in the alternative, may be constructed of two or more layers of materials.

Various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers.

If a spacer layer is present, the absorbent body 34 can be disposed on the spacer layer and superposed over the outer cover 26. The spacer layer can be bonded to the outer cover 26, for example, by adhesive. In some embodiments, a spacer layer may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In some embodiments, at least a portion of a layer, such as but not limited to, a fluid transfer layer and/or a spacer layer, can be positioned between the absorbent body 34 and the outer cover 26. The absorbent body 34 can be bonded to the fluid transfer layer and/or the spacer layer.

### Containment Flaps

In an embodiment, the absorbent article 10, 210 can include a pair of containment flaps 50, 52. The containment flaps 50, 52 can be formed separately from the absorbent chassis 11 and attached to the chassis 11 or can be formed integral to the chassis 11. In some embodiments, the containment flaps 50, 52 can be secured to the chassis 11 of the absorbent article 10, 210 in a generally parallel, spaced relation with each other laterally inward of the leg openings to provide a barrier against the flow of bodily exudates. One containment flap 50 can be on a first side of the longitudinal axis 29 and the other containment flap 52 can be on a second side of the longitudinal axis 29. In an embodiment, the containment flaps 50, 52 can extend generally in a longitudinal direction 30 from the front waist region 12 of the absorbent article 10, through the crotch region 16 to the rear waist region 14 of the absorbent article 10. In some embodiments, the containment flaps 50, 52 can extend in a direction substantially parallel to the longitudinal axis 29 of the absorbent article 10, 210, however, in other embodiments, the containment flaps 50, 52 can be curved, as is known in the art. In other embodiments, such as the absorbent article 210 in FIGS. 3 and 4, the containment flaps 50, 52 can be disposed on the absorbent panel 17 in the crotch region 16.

In embodiments where the containment flaps 50, 52 are coupled to the chassis 11, the containment flaps 50, 52 can be bonded to the bodyside liner 28 with a barrier adhesive. Alternatively, the containment flaps 50, 52 can be bonded to the outer cover 26 with a barrier adhesive, or to the spacer layer. Of course, the containment flaps 50, 52 can be bonded to other components of the chassis 11 and can be bonded with other suitable means other than a barrier adhesive. The containment flaps 50, 52 can be constructed of a fibrous material which can be similar to the material forming the bodyside liner 28. Other conventional materials, such as polymer films, can also be employed.

The containment flaps 50, 52 can each include a base portion 64 and a projection portion 66. The base portion 64 can be bonded to the chassis 11, for example, to the bodyside liner 28 or the outer cover 26 as mentioned above. The base portion 64 can include a proximal end 64a and a distal end 64b. The projection portion 66 can be separated from the base portion 64 at the proximal end 64a of the base portion 64. As used in this context, the projection portion 66 is separated from the base portion 64 at the proximal end 64a of the base portion 64 in that the proximal end 64a of the base portion 64 defines a transition between the projection portion 66 and the base portion 64. The proximal end 64a of the base portion 64 can be located near the barrier adhesive. In some embodiments, the distal ends 64b of the base portion 64 can laterally extend to the respective longitudinal side edges 18, 20 of the absorbent article 10, 210. In other embodiments, the distal ends 64b of the base portion 64 can end laterally inward of the respective longitudinal side edges 18, 20 of the absorbent article 10, 210. The containment flaps 50, 52 can also each include a projection portion 66 that is configured to extend away from the body facing surface 19 of the chassis 11 at least in the crotch region 16 when the absorbent article 10, 210 is in a relaxed configuration. The containment flaps 50, 52 can include a tack-down region 71 in either or both of the front waist region 12 and the rear waist region 14 where the projection portion 66 is coupled to the body facing surface 19 of the chassis 11.

It is contemplated that the containment flaps 50, 52 can be of various configurations and shapes, and can be constructed by various methods. For example, the containment flaps 50, 52 can extend nearly vertically with a tack-down region 71 in both the front and rear waist regions 12, 14 where the projection portion 66 of each containment flap 50, 52 is tacked down to the bodyside liner 28 towards or away from the longitudinal axis 29 of the absorbent article 10, 210. However, the containment flaps 50, 52 can include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 could be constructed in a “T-shape” configuration, such as described in U.S. Pat. No. 9,259,362 by Robert L. Popp et al. Such a configuration can also include a tack-down region 71 in either or both of the front and rear waist regions 12, 14, respectively. Of course, other configurations of containment flaps 50, 52 can be used in the absorbent article 10, 210 and still remain within the scope of this disclosure.

The containment flaps 50, 52 can include one or more flap elastic members 68, such as the two flap elastic strands depicted in FIG. 2. Suitable elastomeric materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastomeric properties itself.

The flap elastic members 68 can have two strands of elastomeric material extending longitudinally in the projection portion 66 of the containment flaps 50, 52, in generally parallel, spaced relation with each other. The elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, 210, when the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, 110% to 350%. In one embodiment, the elastic members 68 can be coated with adhesive while elongated to a specified length prior to attaching to the elastic members 68 to the containment flaps 50, 52. In a stretched condition, the length of the elastic members 68 which have adhesive coupled thereto can provide an active flap elastic region 70 in the containment flaps 50, 52, as labeled in FIG. 2, which will gather upon relaxation of the absorbent article 10. The active flap elastic region 70 of containment flaps 50, 52 can be of a longitudinal length that is less than the length of the absorbent article 10, 210. In this exemplary method of bonding the elastic members 68 to the containment flaps 50, 52, the portion of the elastic members 68 not coated with adhesive, will retract after the elastic members 68 and the absorbent article 10 are cut in manufacturing to form an individual absorbent article 10. As noted above, the relaxing of the elastic members 68 in the active flap elastic region 70 when the absorbent article 10, 210 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28).

Of course, the elastic members 68 can be bonded to the containment flaps 50, 52 in various other ways as known by those of skill in the art to provide an active flap elastic region 70, which is within the scope of this disclosure. Additionally, the active flap elastic regions 70 can be shorter or longer than depicted herein, including extending to the front waist edge 22 and the rear waist edge 24, and still be within the scope of this disclosure.

### Leg Elastics

Leg elastic members 60, 62 can be secured to the outer cover 26, such as by being bonded thereto by laminate adhesive, generally laterally inward of the longitudinal side edges, 18 and 20, of the absorbent article 10, 210. The leg elastic members 60, 62 can form elasticized leg cuffs that further help to contain bodily exudates. In an embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein each include two elastic strands. A wide variety of elastomeric materials may be used for the leg elastic members 60, 62.

Suitable elastomeric materials can include sheets, strands or ribbons of natural rubber, synthetic rubber, or thermoplastic elastomeric materials. The elastomeric materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some embodiments. Of course, the leg elastic members 60, 62 can be omitted from the absorbent article 10, 210 without departing from the scope of this disclosure.

### Waist Containment Member

In an embodiment, the absorbent article 10, 210 can have one or more waist containment members 54. The waist containment member(s) 54 can be disposed in the rear waist region 14 as illustrated in FIGS. 1-4. In general, the waist containment member 54 can help contain and/or absorb bodily exudates, especially low viscosity fecal matter, and as such, can be preferred to be in the rear waist region 14. In some embodiments, the absorbent article 10, 210 can have a waist containment member 54 disposed in the front waist region 12. A waist containment member 54 in the front waist region 12 can help contain and/or absorb bodily exudates, such as urine, in the front waist region 12. Although not as prevalent as in the rear waist region 14, in some circumstances, fecal material may also spread to the front waist region 12, and thus, a waist containment member 54 disposed in the front waist region 12 can help contain and/or absorb bodily exudates as well. In other embodiments, the absorbent article 10, 210 can have a waist containment member 54 in both the rear waist region 14 and the front waist region 12.

The waist containment member 54 can be disposed on the body facing surface 45 of the absorbent assembly 44. In some embodiments, such as in embodiments illustrated in FIGS. 1 and 2, the waist containment member 54 can be disposed on the body facing surface 56 of the bodyside liner 28. However, in some embodiments, such as the absorbent article 210 in FIG. 4, the waist containment member 54 can be disposed on a body facing surface 58 of the rear waist panel 15.

The waist containment member 54 can include a first longitudinal side edge 72 and a second longitudinal side edge 74. The first longitudinal side edge 72 can be opposite from the second longitudinal side edge 74. The distance between the first longitudinal side edge 72 and the second longitudinal side edge 74 can define a width of the waist containment member 54 in the lateral direction 32, as shown in FIG. 2.

As illustrated in FIG. 2, the waist containment member 54 can be configured such that the first longitudinal side edge 72 can be disposed laterally outward of the proximal end 64a of the base portion 64 of the containment flap 50. Similarly, the waist containment member 54 can be configured such that the second longitudinal side edge 74 can be disposed laterally outward of the proximal end 64a of the base portion 64 of the containment flap 52. The waist containment member 54 can be configured such that the width 51 of the waist containment member 54 can be greater than a lateral distance between longitudinal extending fold lines 25a, 25b, as shown in FIGS. 2 and 4.

The waist containment member 54 can also include a proximal portion (not shown) and a distal portion (not shown). The proximal portion can be coupled to the body facing surface 19 of chassis 11 (e.g., the body facing surface 45 of the absorbent assembly 44 or the body facing surface 56 of the bodyside liner 28) whereas the distal portion of the waist containment member 54 can be free to move with respect to the chassis 11 and the absorbent assembly 44 when the absorbent article 10, 210 is in the relaxed configuration. For example, the proximal portion may be folded under the distal portion, with the fold defining a transition between the proximal portion and the distal portion. When the waist containment member 54 is in a relaxed configuration, the distal portion extends away from the chassis 11 and absorbent assembly 44 in a vertical direction, which is perpendicular to the plane defined by the longitudinal axis 29 and the lateral axis 31.

In some embodiments, the proximal portion of the waist containment member 54 can be coupled to the body facing surface 56 of the bodyside liner 28. In other embodiments, the proximal portion of the waist containment member 54 can be coupled to the body facing surface 58 of the rear waist panel 15. The proximal portion can be coupled to the body facing surface 45 by an adhesive, by pressure bonding, by ultrasonic bonding, by thermal bonding, and combinations thereof.

Because the distal portion of the waist containment member 54 can freely move with respect to the absorbent assembly 44 when the absorbent article 10, 210 is in the relaxed configuration, the distal portion can help provide a containment pocket when the absorbent article 10, 210 is in the relaxed configuration. In such configurations, the containment pocket is disposed under the distal portion and between the distal portion and the body facing surface 19 of the chassis 11. The containment pocket can help provide a barrier to contain and/or can help absorb bodily exudates. The containment pocket can be especially beneficial for containing and/or absorbing low viscosity fecal matter, which can be prevalent in younger children. The first longitudinal side edge 72 can be disposed laterally outward of the proximal end 64a of the base portion 64 of the containment flap 50, and thus, the containment pocket can extend laterally outward of the proximal end 64a of the containment flap 50. Similarly, the second longitudinal side edge 74 can be disposed laterally outward of the proximal end 64a of the base portion 64 of the containment flap 52 and the containment pocket can extend laterally outward of the proximal end 64a of the containment flap 52. Such a configuration provides waist containment member 54 with a wide containment pocket 82 to contain and/or absorb bodily exudates.

To help prevent lateral flow of bodily exudates that are contained by the containment pocket of the waist containment member 54, the distal portion of the waist containment member 54 can be bonded to the proximal portion of the waist containment member 54 and/or the body facing surface 19 of the chassis 11 near the first and second longitudinal side edges 72, 74, respectively. For example, FIG. 2 depicts tack-down regions 84 where the distal portion of the waist containment member 54 can be bonded to the proximal portion of the waist containment member 54 and/or the body facing surface 19 of the chassis 11.

In preferred embodiments, the waist containment member 54 can include at least one elastic member and even more elastic members in further embodiments. Generally, the elastic member can span substantially from the first longitudinal side edge 72 to the second longitudinal side edge 74 of the waist containment member 54. The elastic member can be disposed in the distal portion of the waist containment member 54, and preferably, is located near a free edge 88 of the distal portion of the waist containment member 54.

A wide variety of elastomeric materials may be used for the elastic member(s) in the waist containment member 54. Suitable elastomeric materials can include sheets, strands or ribbons of natural rubber, synthetic rubber, elastic foams, or thermoplastic elastomeric materials (e.g., films). The elastomeric materials can be stretched and secured to a substrate forming the waist containment member 54, secured to a gathered substrate, or secured to a substrate and then elasticized or shrunk, for example, with the application of heat, such that the elastic retractive forces are imparted to the substrate forming the waist containment member 54.

The waist containment member 54 can be disposed to be coupled to the chassis 11 by being placed either over the containment flaps 50, 52 or under the containment flaps 50, 52. More specifically, the waist containment member 54 can be disposed on the body facing surface 19 of the chassis 11 such that the proximal portion of the waist containment member 54 is disposed over the base portion 64 of the first and the second containment flaps 50, 52, respectively. Alternatively, the waist containment member 54 can be disposed on the body facing surface 19 of the chassis 11 such that the proximal portion of the waist containment member 54 is disposed under the base portion 64 of the first and the second containment flaps 50, 52, respectively. Both configurations can provide advantages to the functioning of the waist containment member 54 to contain and/or absorb bodily exudates.

Where the proximal portion of the waist containment member 54 is disposed over the base portion 64 of the containment flaps 50, 52, the containment flaps 50, 52 can have an active flap elastic region 70 that longitudinally overlaps with the distal portion 78 of the waist containment member 54 when the absorbent article 10 is in the stretched, laid flat configuration, such as illustrated in FIG. 2. Additionally or alternatively, the tack-down region 71 may not extend from the rear waist edge 24 to the free edge 88 of the distal portion of the waist containment member 54, such as illustrated in FIG. 2.

Where the proximal portion of the waist containment member 54 is disposed under the base portion 64 of the containment flaps 50, 52, the tack-down region 71 of the projection portion 66 of each of the containment flaps 50, 52 may longitudinally overlap with the distal portion of the waist containment member 54. In some of these embodiments, the tack-down region 71 of projection portion 66 of each of the containment flaps 50, 52 can extend to the free edge 88 of the waist containment member 54 to further assist in containing exudates to the containment pocket created by the waist containment member 54.

The waist containment member 54 can be comprised of a variety of materials. In a preferred embodiment, the waist containment member 54 can be comprised of a spunbond-meltblown-spunbond (“SMS”) material. However it is contemplated that the waist containment member 54 can be comprised of other materials including, but not limited to, a spunbond-film-spunbond (“SFS”), a bonded carded web (“BCW”), or any non-woven material. In some embodiments, the waist containment member 54 can be comprised of a laminate of more than one of these exemplary materials, or other materials. In some embodiments, the waist containment member 54 can be comprised of a liquid impermeable material. In some embodiments, the waist containment member 54 can be comprised of a material coated with a hydrophobic coating. The basis weight of the material forming the waist containment member 54 can vary, however, in a preferred embodiment, the basis weight can be between 8 gsm to 120 gsm, not including the elastic members 86 in the waist containment member 54. More preferably, the basis weight of the material comprising the waist containment member 54 can be between 10 gsm to 40 gsm, and even more preferably, between 15 gsm to 25 gsm.

### Fastening System

In an embodiment, the absorbent article 10 can include a fastening system. The fastening system can include one or more back fasteners 91 and one or more front fasteners 92. The embodiments shown in FIGS. 1 and 2 depict embodiments with one front fastener 92. Portions of the fastening system may be included in the front waist region 12, rear waist region 14, or both.

The fastening system can be configured to secure the absorbent article 10 the waist of the wearer in a fastened condition as shown in FIG. 1 and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 2. As shown in FIG. 2, in some embodiments the waist containment member 54 can extend to back fasteners 91. In some embodiments, the waist containment member 54 can be coupled to the stretch component 94 of the back fasteners 91, either directly or indirectly. In some embodiments, the waist containment member 54 can extend to the longitudinal side edges 18, 20 of the absorbent article 10, 210.

### Dual Zoned Web

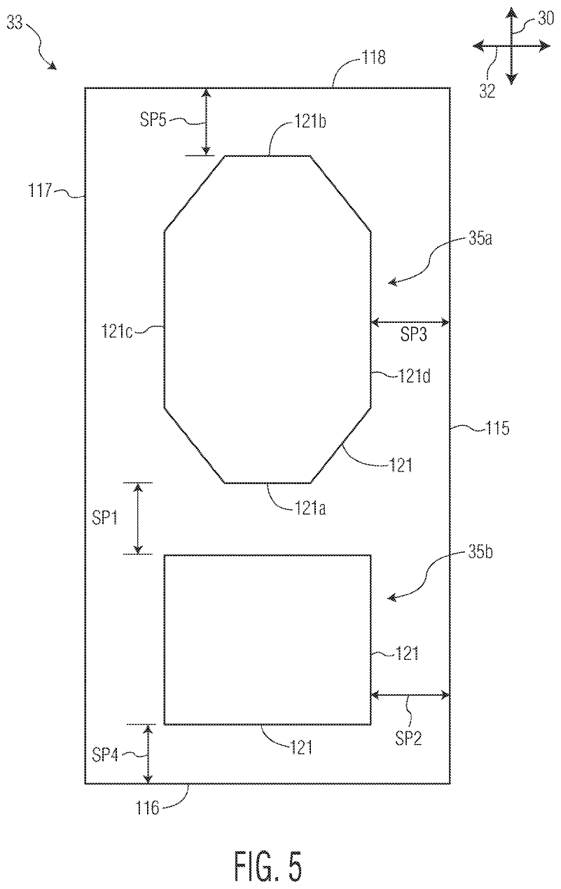

In some embodiments, the absorbent article 10, 210 can further include multi-zoned web 33 one example of which is shown in FIG. 5. As described herein, the multi-zoned web 33 may be described as a dual-zoned web 33 having a first zone 35a and a second zone 35b. However, it should be understood that such multi-zoned webs 33 according to aspects of the present disclosure are not limited to just a first zone 35a and a second zone 35b. Rather, such multi-zoned webs 33 can have greater than two zones, such as three zones, four zones, five zones, or even more zones without departing from the scope of the present disclosure.

Dual-zoned web 33 according to some embodiments may be a topical liner material coupled to the body facing surface 19 of the bodyside liner 28. The first zone 35a and the second zone 35b can be visually distinct and individually contiguous regions of the dual-zoned web 33. Each of the zones 35a, 35b comprise one or more morphological features which have been imparted onto to the web 33 by one or more processes to form such morphological features. Example morphological features include embossments, apertures, and/or protrusions. The zones 35a, 35b are individually contiguous containing respective morphological features and are surrounded by regions having no morphological features or morphological features separate and distinct from those morphological features forming the zones 35a, 35b. In this manner, the zones 35a, 35b have clear outer boundaries formed at least in part by their respective morphological features.

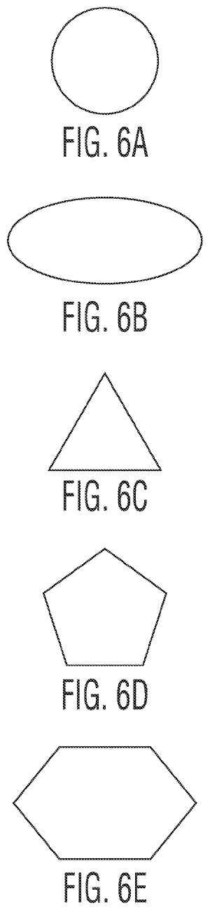

According to some aspects of the present disclosure, the first zone 35a and the second zone 35b may have different shapes defined by their outer boundaries. For example, the outer boundaries of the first zone 35a and/or the second zone 35b may define a square shape (as shown for second zone 35b in FIG. 5) or an octagon shape (as shown for first zone 35a in FIG. 5). Although, it should be understood that the zones 35a, 35b may form other polygonal shapes such as triangles, pentagons, hexagons, and the like. In still further examples, the zones 35a, 35b may be circular or oval shaped. Example shapes for the zones 35a, 35b are shown in FIGS. 6A-6E.

As will be described in more detail below, the zones 35a, 35b may be configured differently to handle different bodily exudates effectively. At least some examples include where the zones 35a, 35b have different shapes in order to help communicate such a difference in functionality of the zones 35a, 35b. As seen in FIG. 5, the first zone 35a may have an octagon shape while the second zone 35b has a square shape. Although, this is just one example. Other examples can include the first zone 35a having any of the above-described shapes, and the second one 35b having any of the above-described shapes while having a different shape from the first zone 35a. Employing such different shaping for the different zones 35a, 35b helps to highlight the differences in functionality of the zones 35a, 35b. In particular, it may be desired for the zone configured to handle BM exudates to be ‘target’-shaped—such as being a circle or having an oval, pentagon, hexagon, or octagon shape. In such examples, particularly in conjunction with such a zone being located in a rear portion of an absorbent article, this target shape helps to communicate the effectiveness of the zone for handling BM exudates.

Another functional benefit of forming one or more of the zones 35a, 35b in a circle, an oval shape, or a polygon shape other than a square is that the total area of the zones 35a, 35b may be reduced relative to forming the zones 35a, 35b as squares. Particularly where the imparted morphological features of the zones 35a, 35b are apertures, such morphological features decrease the overall web strength and/or create different zones of strength across the transverse direction 32 (e.g. the cross-direction or CD) of the web 33. This decreased strength and/or differentiated strength in the CD has impacts to web handling and processability. Accordingly, utilizing such non-square shapes for at least one of the zones 35a, 35b helps to reduce the decreased or differentiated strength in the CD of the web 33 thereby improving the handling and processability of the web 33 relative to forming both of the zones 35a, 35b as square shapes.

Where one of the zones 35a, 35b is a pentagon, hexagon, or octagon, such zones 35a, 35b may be disposed on the web 33 such that an outer boundary in disposed extending in the transverse direction 32. Outer boundary 121a of zone 35a in FIG. 5 is shown oriented in such a direction. By orienting at least one of the outer boundaries 121 of the zone 35a in this manner, the zone 35a maintains a target-like appearance and helps to convey the functional nature of the zone 35a—in contrast to configurations where such outer boundaries are all skewed with respect to the transverse direction 32. Where the zone 35a has an octagon shape, it can be seen that orienting the outer boundary 121a in the transverse direction 32 has the effect of orientating the outer boundary 121b in the transverse direction 32 as well, along with orienting the outer boundaries 121c,d in the longitudinal direction 30. As shown in FIG. 5, the outer boundaries 121 of the zones 35a, 35b define the perimeter of the zones 35a, 35b. It should be understood that such outer boundaries 121 defining the perimeter of the zones 35a, 35b are formed by the extent of the morphological features of the zones 35a, 35b. Accordingly, the zones 35a, 35b may not have strict linear portions defining the perimeter as shown with outer boundaries 121. However, such shapes as described with respect to FIGS. 6A-6E (along with square shapes) may be readily discernable from the presence-to-absence of the morphological features of the zones 35a, 35b at their perimeters in webs 33 according to aspects of the present disclosure.

It may be particularly important in some embodiments for the outer boundaries 121a and/or 121b to be oriented in the transverse direction 32 as well as to have at least four (4) aperture morphological features extending in the transverse direction 32 proximate the outer boundaries 121a and/or 121b. Where the web is dual-zoned, such as in FIG. 5, it may be important to be able to register the web 33 appropriately within an absorbent article such as article 10. To accomplish such registration, it may be desirable to be able to identify particular features of the web 33 at high speeds. Ensuring that at least one of the outer boundaries 121a and/or 121b is oriented in the transverse direction 32 and that there are at least four (4) aperture morphological features extending in the transverse direction 32 proximate the outer boundaries 121a and/or 121b provides such features which are able to be identified by registration equipment at high speeds. In further embodiments, particularly where the aperture morphological features are small, it may be desirable that at least six (6), at least eight (8), or at least ten (10) aperture morphological features are positioned extending in the transverse direction 32 proximate the outer boundaries 121a and/or 121b.

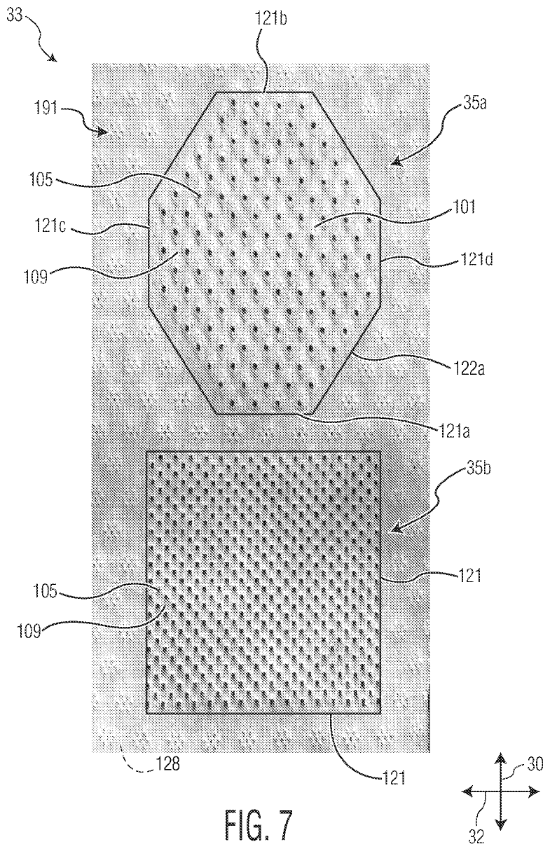

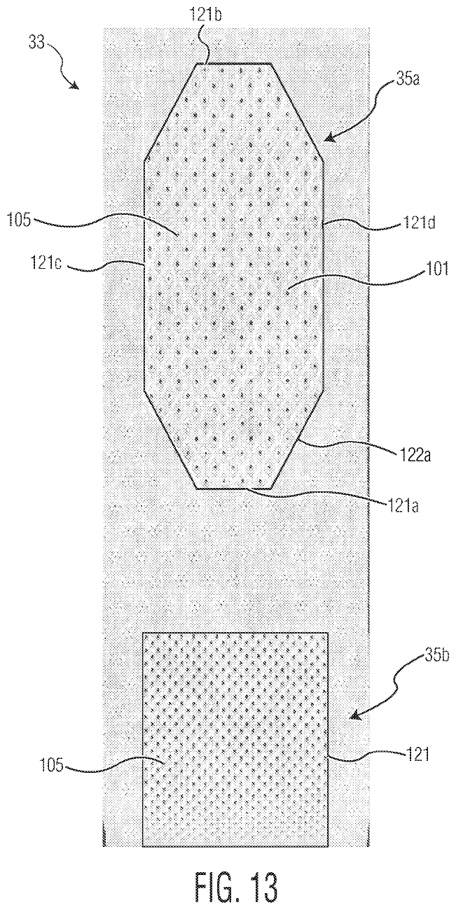

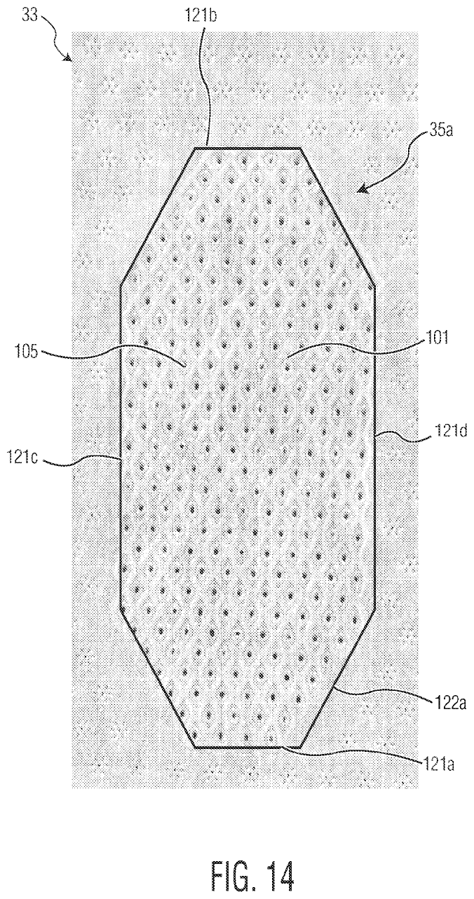

In some further embodiments, it may be desirable to keep the lengths of the outer boundaries of the zones 35a, 35b oriented in the longitudinal direction 30, for instance outer boundaries 121c,d, equal to or less than lengths of the outer boundaries of the zones 35a, 35b oriented in a direction skewed to both the longitudinal direction 30 and the lateral direction 32, such as outer boundary 122a. Such embodiments are shown with respect to web 33 of FIG. 7, which is an optical image of an exemplary web 33 according to aspects of the present disclosure. Although in other embodiments, in order to ensure a sufficient coverage of the first or second zones 35a, 35b on the web 33—for instance to ensure capture of the body exudates by the first and/or second zones 35a, 35b—it may be preferable for the lengths of the outer boundaries of the zones 35a, 35b oriented in the longitudinal direction 30 (e.g. outer boundaries 121c,d) to have a length that is longer than lengths of the outer boundaries of the zones 35a, 35b oriented in a direction skewed to both the longitudinal direction 30 and the lateral direction 32 (e.g. outer boundary 122a). Such a configuration is depicted in FIGS. 13 and 14. FIG. 13 is an optical image of an exemplary web 33 according to aspects of the present disclosure having a first zone 35a and a second zone 35b, while FIG. 14 is a close-up of the first zone 35a of the web 33 of FIG. 13.

One aspect of the zones 35a, 35b of the web 33 is the relative sizes of the zones. The sizes of the zones 35a, 35b may be chosen as a balance between being large enough to handle the bodily exudates but not so large as to impact the web handling and processability of the web 33 to such a degree that it is unable to be processed in a high-speed manufacturing process for incorporation into an absorbent article. As shown with respect to further figures, aspects of the present disclosure include the first zone 35a being configured to efficiently handle BM exudates and the second zone 35b being configured to efficiently handle urine exudates. In such embodiments, the first zone 35a may have a total area of between 3,000 mm² and 9,000 mm². In other embodiments, the first zone 35a may have a total area of between 3,000 mm² and 8,500 mm², between 3,500 mm² and 8,000 mm², or between 4,000 mm² and 7,750 mm². The second zone 35b may have a total area of between 3,000 mm² and 6,000 mm², between 3,500 mm² and 5,500 mm², or between 4,000 mm² and 5,000 mm². A total area of the web 33 may be between 15,000 mm² and 30,000 mm², between 17,500 mm² and 27,500 mm², or between 17,500 mm² and 25,000 mm².

Additionally, in order to ensure processability of the web 33 in a high-speed manufacturing process, the zones 35a, 35b may have particular spacings from each other and from other portions of the web 33. For instance, the zones 35a, 35b should be spaced from each other, as detailed by distance SP1 in FIG. 5, of between 7.5 mm and 65 mm, between 10 mm and 55 mm, or between 10 mm and 40 mm. These ranges allow for both sufficient spacing between the zones 35a, 35b to allow for differentiation of the zones 35a, 35b with respect to registration of the material during a manufacturing process as well as ensuring that the zones 35a, 35b are positioned desirably with respect to appropriate insult locations for the separate urine and BM insults to be handled by the zones 35a, 35b.

As well, the zones 35a, 35b can be spaced particular distances from the longitudinally extending side edges 115, 117 and laterally extending end edges 116, 118 of the web 33. Such distances that the zones 35a, 35b are spaced from the longitudinally extending side edges 115, 117 are labeled as SP2 and SP3 in FIG. 5. For instance, it has been found that the zones 35a, 35b are desirably spaced from the longitudinally extending side edges 115, 117 by at least 5 mm—e.g. SP2 and SP3 have values of at least 5 mm. In other embodiments, the zones 35a, 35b are desirably spaced from the longitudinally extending side edges 115, 117 by at least 7.5 mm, or at least 10 mm, or at least 15 mm, or at least 25 mm. Such distances help to ensure sufficient MD tensile strength across the entire CD span of the web 33 with such MD tensile strength being impacted (e.g. reduced) by the morphological features of the zones 35a, 35b. As well, it has been found that sufficient widths of the web 33 are needed on the sides of the zones 35a, 35b to reduce curling effects of the web 33 when placed under MD tension, such as during high-speed manufacturing processes.

Additionally, the zones 35a, 35b are desirably spaced from the longitudinally extending side edges 115, 117 by less than 75 mm—e.g. SP2 and SP3 have values of less than 75 mm. In further embodiments, the zones 35a, 35b are desirably spaced from the longitudinally extending side edges 115, 117 by less than 60 mm, or by less than 50 mm, or by less than 40 mm, or by less than 35 mm. It is desired for the spacing between the zones 35a, 35b and the longitudinally extending side edges 115, 117 to not be too large to ensure no interference between of the web 33 with bonding of the flaps 50, 52 and the bodyside liner 28. Accordingly, the desired spacing SP2 and SP3 are between 5 mm and 75 mm, or between 5 mm and 60 mm, or between 5 mm and 50 mm, or between 7.5 mm and 40 mm.

The spacings SP4 and SP5 are less critical in terms of performance of the web 33 than the spacings SP2 and SP3. Although, it may be important for the spacings SP4 and SP5 to be between 5 mm and 75 mm, or between 5 mm and 60 mm, or between 10 mm and 50 mm, or between 10 mm and 40 mm.

FIG. 7 depicts one exemplary configuration of a dual-zoned web material 33 according to aspects of the present disclosure. As can be seen with respect to FIG. 7, the web 33 has a first zone 35a forming an octagon shape and a second zone 35b forming a square shape (with outer boundaries 121 drawn to more easily visualize the shape of zones 35a, 35b). The web 33 of FIG. 7 further depicts various morphological features of the zones 35a, 35b. As should be understood, the zones of any of the web materials of the present disclosure may have one or more morphological features as depicted in the web material 33 of FIG. 7. For instance, web materials according to aspects of the present disclosure may have morphological features such as embossments 101, apertures 105, and/or protrusions 109. Embossments 101 can be depressions on a first surface of the web 33 without causing a corresponding protrusion on the opposite surface of the web 33. Apertures 105 are generally openings in the web material sufficient for fluids to pass through un-encumbered by the fibers of the web 33. Such apertures 105 are distinguished from the interstitial fiber-to-fiber spacing of web materials which may provide some irregular, disordered openings within the web 33, typically varying as a function of basis weight of the web material 33. Protrusions 109 are morphological features of the web 33 whereby a portion of the web 33 is raised above a surface of the web 33-the surface being defined by un-modified regions of the web 33.

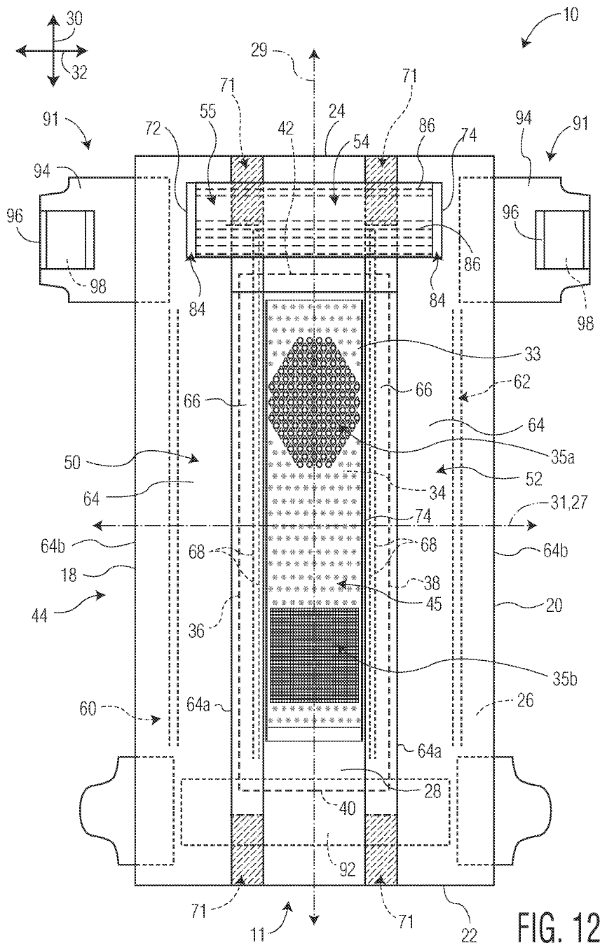

FIG. 12 is a schematic drawing of an exemplary web material 33 according to aspects of the present disclosure and having features similar to those depicted in FIG. 7 disposed within an absorbent 10. The web 33 of FIG. 12 may be the bodyside liner material 28 of the article 10 with the zones 35a, 35b of the web 33 disposed as shown in relation to other components of the article 10. In alternative embodiments, the web 33 may be a topical liner material coupled to a bodyside liner material 28. For instance, the web 33 may be coupled to a body facing surface of the bodyside liner material 28. In any such embodiments, it may be advantageous for the zones 35a, 35b to be disposed in particular relations to other components of the article 10. According to some aspects of the present disclosure, the zones 35a and/or 35b may be disposed such that outer boundaries of the zones 35a, and/or 35b are disposed entirely between the projection portions 66 of the containment flaps 50, 52 when the article 10 is disposed in a stretched and laid flat configuration. Further, it may be beneficial for the first zone 35a to be disposed in a rear portion of the article 10, for example between the lateral axis 31 of the article 10 and the rear waist edge 24. The second zone 35b may preferably be disposed in a front portion of the article 10, for example between the lateral axis 31 of the article 10 and the front waist edge 22. It may also be beneficial for the zones 35a and/or 35b of the web 33 to be entirely disposed within the confines of the absorbent body 34., as shown in FIG. 12. This ensures that the uptake of the bodily exudates captured by the zones 35a and/or 35b to be efficiently transported into and handled by further interior components of the article 10.

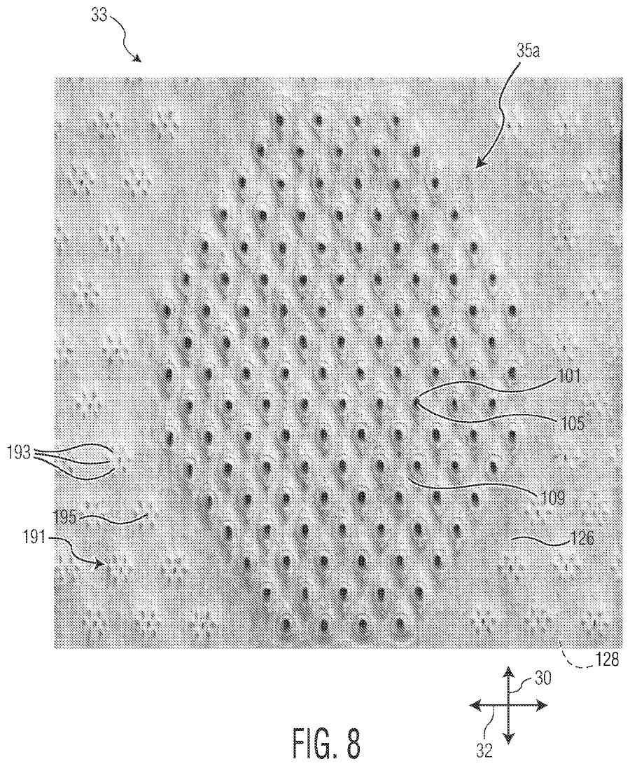

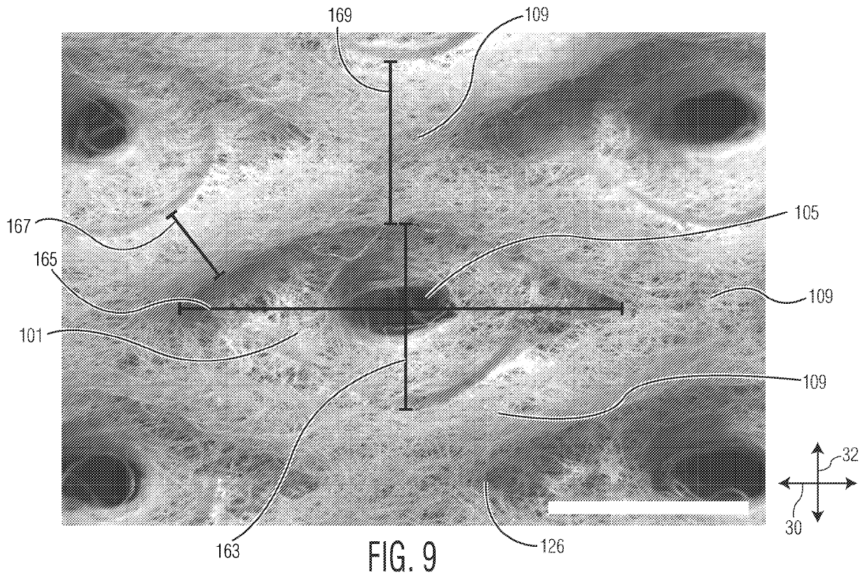

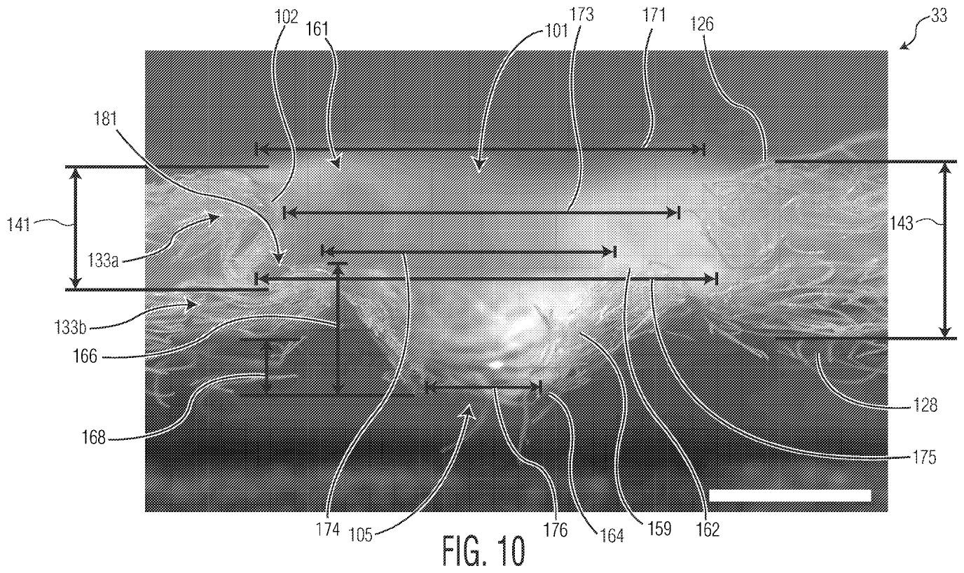

As described previously, at least one of the zones 35a, 35b may be configured to handle BM exudates efficiently. As described with respect to web 33 of FIG. 7, the first zone 35a may be the zone configured to handle BM exudates. In such embodiments, the first zone 35a may comprise embossments 101, with the embossments 101 forming depressions in a first surface of the web 33. FIG. 8 is a close-up of a portion of the first zone 35a of FIG. 7 to understand aspects of the first zone 35a in more detail. FIG. 9 is a close-up image captured by a stereo microscope of exemplary embossments 101 of the zone 35a, while FIG. 10 is a close-up image captured by a stereo microscope of a cross-section of an exemplary embossment 101 of the first zone 35a.

Preferably, the embossments 101 may have a depth 141 (as shown in FIG. 10) of between 0.8 mm and 2.5 mm or between 0.9 mm and 2.25 mm, or between 1.0 mm and 2.0 mm. The depth 141 may in alternative embodiments be described as a percentage of the thickness of the material 33. According to such a description, the embossments 101 may have a depth 141 that is greater than 50% of a thickness 143 of the web 33 in un-modified regions—e.g. regions of the web 33 that do not contain morphological features imparted on the web 33. In further embodiments, the depth 141 may be between than 50% and 80% or between 50% and 70% of the thickness 143.

The embossments may desirably have areas of between 20 mm² and 40 mm². In alternative embodiments, the embossments may have areas of between 23 mm² and 37 mm² or between 25 mm² and 35 mm². Such areas may desirably provide for sufficient depressed volumes to collect BM exudates away from a wearer's skin prior to the exudates flowing through the web 33 and into an interior of an absorbent article utilizing a web such as web 33. Such volumes have been found to help prevent skin irritation by providing sufficient volumes for temporary storage of the BM exudates and preventing spread of the BM exudates to minimize skin area which contacts the BM exudates. Where the embossments 101 have too large of an area, spread of the BM exudates is increased due to a decrease in the barriers to flow (e.g. the protrusions 109 disposed between embossments 101).

At least some embodiments of the present disclosure include where the embossments have a diamond shape, as can be seen most clearly in FIGS. 8-9. In such embodiments, it may be preferable for the width 163 of the embossments 101 in the transverse direction 32 to be less than their lengths 165 in the longitudinal direction 30. For example, such configurations increase the frequency of the protrusions 109 that BM exudates encounter as they spread in the transverse direction 32, thereby limiting the spread of the BM exudates in the transverse direction relative to the longitudinal direction 30. Such a configuration is preferred to decrease ability of BM exudate to flow in the transverse direction 32 and encounter the containment flaps (such as flaps 50, 52 of articles 10, 210), which can be a point of leakage for an absorbent article where the BM contacts such containment flaps with sufficient force. Accordingly, in these embodiments, the length 165 of the embossment 101 in the longitudinal direction 30 may be between 1.5 to 3.0 times greater than the width 163 of the embossment 101 in the transverse direction 32.

Particularly in combination with the above-described features, webs 33 according to aspects of the present disclosure may have a total number of embossments of between 100 and 300, or between 115 and 275, or between 125 and 250. Such numbers of embossments 101 can produce embossed areas of between 3,000 mm² and 10,000 mm², or between 3,500 mm² and 8,000 mm². Described a different way, it is preferred for the embossed area of the first zone 35a to be between 65% and 95% of the total area of the zone 35a or between 70% and 90% of the total area of the zone 35a, or between 75% and 85% of the total area of the zone 35a. Such a high embossed-percentage ensures a high amount of volume for retaining BM exudates away from a wearer's skin.

In at least some embodiments, the first zone 35a may further comprise apertures 105. In order to efficiently handle BM exudates, the apertures 105 may each preferably have areas of between 0.5 mm² and 10 mm². In alternative embodiments, the apertures 105 may each have areas of between 0.5 mm² and 8 mm², between 0.5 mm² and 6 mm², between 0.75 mm² and 5 mm², between 0.75 mm² and 4 mm², or between 1.5 mm² and 4 mm². In further embodiments, it may be desirable for the apertures 105 to each preferably have larger areas, such as between 6 mm² and 20 mm², 8 mm² and 18 mm² or between 10 mm² and 15 mm². In at least some of these embodiments, the apertures may be circle shape or oval shaped to help speed flow of the BM exudates through the web 33. Where the apertures 105 are oval shaped, the apertures may have a major axis and a minor axis. Preferably, the major axis is oriented approximately in the longitudinal direction 30. Particularly in conjunction with embossments 101 having lengths in the longitudinal direction 30 greater than widths in the transverse direction 32, such orientations of the apertures 105 helps to channel BM exudates which are preferentially guided in the longitudinal direction 30 more efficiently through such apertures 105 and through the web 33. The major axis may preferably be about 50% to 200% larger than the minor axis or between 75% and 150% larger than the minor axis.

It has been found that in conjunction with the above-described features, it is beneficial for the first zone 35a to have a total apertured area of between 90 mm² and 2,000 mm². In other embodiments, the total apertured area may be between 100 mm² and 1,500 mm², or between 200 mm² and 1000 mm². Described a different way, it is preferred for the apertured area of the first zone 35a to be between 2.5% and 14% of the total area of the zone 35a or between 3% and 12%, or between 3% and 10%, or between 3% and 8% of the total area of the zone 35a. Such apertured area ranges strike a balance between providing sufficient open area for BM exudates to pass through the web material 33 and maintaining a structural integrity of the material 33 to allow for processability in high-speed manufacturing operations.

Another aspect of the embossments 101 of the first zone 35a is the shape of the embossments 101 in a depth direction perpendicular to the longitudinal and transverse directions 30, 32. As can be seen most clearly with respect to FIG. 10, the embossments 101 of the first zone 35a comprise an opening 161 proximate the first surface 126 of the web 33 and extend into the material 33 for a depth 141. The opening 161 can be seen having a first width 171. With respect to an interior region of the embossment 101, for example as bounded by embossment walls 102, the embossment 101 has a minimum interior width 173 and a maximum interior width 175. In at least some embodiments, it is beneficial for the minimum interior width 173 to be disposed between the maximum interior width 175 and the opening 161. That is, the minimum interior width 173 of the embossment 101 is disposed closer to the first surface 126 of the web 33 than the maximum interior width 175. In such configurations, the embossments 101 may provide for a desirable volume for temporary storage of BM exudates while maintaining a lower surface area of contact with a wearer's skin. For example, BM exudates can be temporarily stored or trapped between the portion of the embossment 101 having the minimum interior width 173 and the portion of the embossment 101 having the maximum interior width 175 which is disposed further away from the opening 161 than the portion of the embossment 101 having the minimum interior width 173. In this way, the portion of the embossment 101 having the minimum interior width 173 can cover at least a portion of the BM exudates within the embossment 101 and preventing them from being in close contact with a wearer's skin and thereby helping to maintain skin health of the wearer.

The first zone 35a of the web 33 may further comprise protrusions 109 extending from the first surface 126 of the web 33, in an opposite direction of the embossments 101. Such protrusions 109 can best be seen in FIG. 9 extending between the embossments 101. Such protrusions 109 can protrude outward from the surface 126 of the web 33 as compared to unmodified portions of the web 33 for a distance of between 0.1 mm and 0.5 mm, or between 0.2 mm and 0.5 mm, or between 0.3 mm and 0.5 mm. Additionally, in order to help prevent spread of BM exudates insulted upon the first zone 35a, such protrusions 109 extending from the first surface 126 can preferably have widths 167 of between 1.5 mm and 2.25 mm, or between 1.7 mm and 2.0 mm, or between 1.75 mm and 1.95 mm between closest adjacent embossments 101. The embossments 101 may be preferentially spaced in the longitudinal direction 30 of the web 33 a distance of between 3.5 mm and 5.0 mm, or between 3.75 mm and 4.5 mm between embossments 101 provided in a same longitudinal column. As well, the embossments 101 may be preferentially spaced in the transverse direction 32 a distance 169 of between 3.5 mm and 4.5 mm, or between 3.75 mm and 4.25 mm between embossments 101 provided in a same transverse row, again providing the protrusions 109 between such embossments 101 these same dimensions. It should be understood that in other embodiments, the protrusions 109 may not protrude at all and instead simply be land areas between the embossments 101 at the same height as the rest of the unmodified portions of the web 33. Even where the web 33 employs such land areas as opposed to protrusions, such land areas having dimensions in the transverse and longitudinal directions 32, 30 and spacings as described above are still useful in decreasing a spread of BM exudates insulted upon the first zone 35a.

In at least some embodiments, the embossments 101 may include protrusions 159 disposed within the embossments 101. These protrusions 159 may extend from an interior of the embossments 101 and outward of a second surface 128 of the web 33. The protrusion 159 may extend beyond the second surface 128 for a distance 168 between 0.5 mm and 1.0 mm. In other embodiments, the protrusion 159 extend beyond the second surface 128 for a distance 168 between 0.65 mm and 0.85 mm. The protrusion 159 may have a proximal base 162 and a distal end 164 and a depth dimension 166 extending between the proximal base 162 and the distal end 164. The depth dimension 166 may be between 0.7 mm and 1.25 mm. Alternative embodiments may have the depth dimension 166 be between 0.75 mm and 1.2 mm or between 0.8 mm and 1.15 mm.

As can be best seen with respect to FIG. 10, at least some of the protrusions 159 have their proximal base 162 disposed closer to the first surface 126 than the bottom of the embossment 101. For example, a distance from the opening 161 of the embossment 101 to the proximal base 162 of the protrusions 159 may be less than the maximum depth dimension 141 of the embossment 101. In this manner, the protrusion 159 and the embossment 101 can form pockets 181 where BM exudates may pool and be isolated from forces action on the protrusions 159. For example, compressive forces on the protrusions 159 from the second surface 128 of the material 33 will tend to push the protrusions 159, including the proximal base portions 162, toward the first surface 126. Where the maximum depth 141 of the embossments 101 is greater than a distance from the opening 161 to the proximal base portions 162 of the protrusions 159, these forces translate to the BM exudates within the embossment 101 where they can be pushed toward a wearer's skin. However, where the distance from the opening 161 of the embossment 101 to the proximal base 162 of the protrusions 159 is less than the maximum depth dimension 141 of the embossment 101, forces pushing the protrusions 159 toward the first surface 126 will not translate to BM exudates disposed at a position further away from the first surface 126 than the proximal base 162 of the protrusions 159. In this manner, BM exudates may be held away from a wearer's skin while inside embossments 101.

Further, in some embodiments, at least some of the protrusions 159 may have their proximal base 162 disposed closer to the first surface 126 than the portion of the embossment 101 having the maximum interior width 175. Such protrusions 159 may further have their proximal base 162 closer to the second surface 128 than the portion of the embossment 101 having the minimum interior width 173. In this manner, the protrusion may help to form the pockets 181 for temporary storage of BM exudates within the embossments 101 where the skin of a wearer may be protected from such temporarily stored BM by the overhang of the portion of the embossment 101 having the minimum interior width 173. Further, by having the portion of the embossment 101 having the minimum interior width 173 disposed closer to the first surface 126 than the proximal base 162 of the protrusion 159, this ensures a sufficient opening between the embossment walls 102 and the proximal base 162 of the protrusion 159 for the BM exudates to enter under the portion of the embossment 101 having the minimum interior width 173. In addition to helping shield BM exudates from upward forces on the protrusions 159, the temporary storage offered by the pockets 181 can be useful where the speed and volume of the BM insult is sufficiently large such that the web 33 cannot transfer the volume of BM away from the skin—for example through apertures 105—fast enough.

The protrusions 159 extending from within the embossments 101 of the first zone 35a may have a funnel-shape in at least some embodiments. Such a funnel shape helps to encourage flow of BM exudates through the protrusions 159 and ultimately the apertures 105 defined by the distal ends 164 of the protrusions 159. Additionally, such funnel shapes help to prevent BM exudates from travelling back through the protrusions 159 and into the embossments 101 and thereby proximate a wearer's skin. Forces on the articles 10, 210 such as the movement of a wearer creating pressure within the article and/or absorbent body can cause pressure within the articles 10, 210 tending to push BM exudates from within the articles 10, 210 toward a body facing surface 19 of the articles 10, 210. In order to efficiently allow BM exudates to flow through the protrusions 159 and corresponding apertures 105 while preventing BM exudates to flow back up through the apertures 105 and into the embossments 101, it has been found to be desirable for the protrusions 159 to have an opening width at their proximal end 162 and a distal end width defining the apertures 105 which narrows such that the distal end width (in the transverse direction 32) is between 30% and 50% of the width of the proximal end opening of the protrusions, or between 35% and 45% in other embodiments.

As will be discussed in more detail below, the web 33 may be formed of two discrete precursor webs in some embodiments. Such precursor webs can be seen in FIG. 10, with respect to precursor web 133a and 133b. In such embodiments, it can be beneficial for the fibers of both of the precursor webs 133a, 133b to form at least a portion of the protrusion 159. By combining fibers of both of the precursor webs 133a, 133b to form the protrusion 159, the protrusion 159 provides a relatively smooth path for the exudates to flow through the web 33 from the first surface 126 and out of the web 33 past the second surface 128.

Conversely, it may be beneficial for fibers of only the precursor web 133a to form the portion of the embossment 101 having the minimum interior width 173. Having lower amounts of fibers in such a portion of the embossment 101 allows for a softer feeling of the first surface 126 of the web 33, which faces a wearer's skin. Additionally, with the fibers of the precursor webs 133a, 133b being relatively uncoupled in such a portion of the embossment 101 allows for greater pliability and/or flexibility of the embossment 101 in this region. This greater pliability and/or flexibility can allow the portion of the embossment 101 to become compressed more easily—for example, by a force from a wearer. As this portion of the embossment 101 is compressed, it will tend to extend toward a center of the embossment 101, thereby shortening the distance 173 in comparison to when the embossment 101 is uncompressed. In this manner, compressive forces on the embossment 101 in such embodiments can thus cause a larger area of the embossment 101 to be covered by the sidewalls 102 of the embossment. As can be seen, such a feature can drive a greater coverage and blocking of BM exudates that are within such embossments 101.

With respect to the portion of the embossment 101 having the maximum interior width 175, it may be desirable for fibers of both of the precursor webs 133a, 133b to be present. The combination of the webs 133a, 133b at these junctions increases the strength of the web 33 and helps to prevent collapse of the protrusions 159. Maintaining the protrusions 159 and the extent that they protrude from the second surface 128 of the web 33 helps to maintain void volume underneath of the web 33 where the web 33 is used as part of an absorbent article. Such void volume is beneficial to provide space for the BM exudates to flow through the material 33 and away from a wearer's skin.

As discussed, the web 33 is generally configured to efficiently handle both urine and BM exudates. Accordingly, with the first zone 35a configured to efficiently handle BM exudates, the second zone 35b may generally be configured to handle urine exudates. As urine and BM exudates are different, their requirements for efficient handling are different as well. Accordingly, the second zone 35b is configured differently than the first zone 35a.

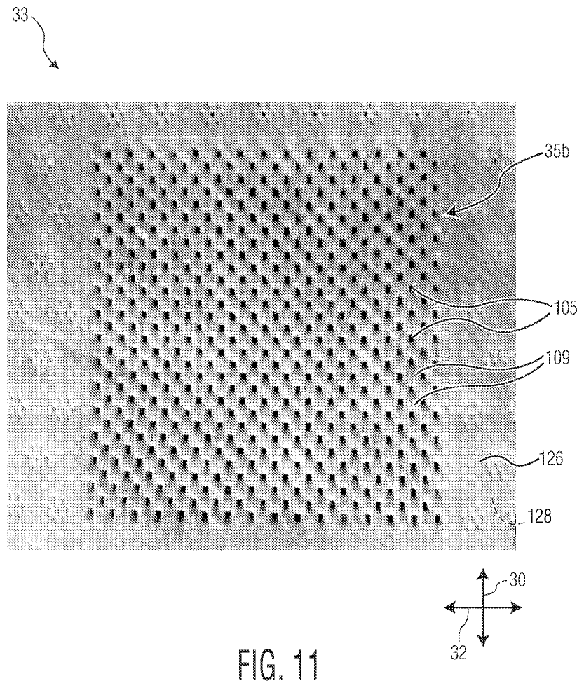

According to some aspects of the present disclosure, the second zone 35b of the web 33 comprises both apertures 105 and protrusions 109. FIG. 11 is a close-up of the first zone of the optical image of FIG. 7. The protrusions 109 of the second zone 35b are oriented to protrude from the second surface 128 of the web 33. Such protrusions may protrude from the second surface 128 for a distance of between 0.25 mm and 1.0 mm, or between 0.4 mm and 0.9 mm, or between 0.5 mm and 0.8 mm. In some embodiments of the web 33, the portions of the web 33 between the protrusions 109 may protrude upward slightly away from the first surface 126 of the web 33 and opposite the protrusions 109 which protrude from the second surface 128 of the web 33. In such embodiments, these protrusions 109 which protrude from the first surface 126 may protrude for a smaller distance than the protrusions 109 which protrude from the second surface 128. For example, such protrusions 109 which protrude from the first surface 126 may protrude for a distance of between 0.1 mm and 0.5 mm, or between 0.1 mm and 0.3 mm.

The protrusions 109 of the second zone 35b may desirably have apertures located within. For example, the protrusions may form an opening at a proximal end in the first side 126 of the web 33 which extends to the distal end of the protrusion 109 of the zone 35b, forming apertures 105. Such apertures 105 may have a variety of shapes in different embodiments. For example, the apertures 105 of the second zone 35b can be circular, have an oval shape, or may be square or rectangular.

The second zone 35b may have a number of apertures 105 of between 300 and 600, or between 350 and 575, or between 400 and 550. The apertures 105 may have areas of between 0.5 mm² and 3.0 mm², or between 0.7 mm² and 2.75 mm², or between 1.0 mm² and 2.0 mm². Such areas and numbers of apertures within the second zone 35b can provide for a total aperture area within the second zone 35b of between 200 mm² and 2,250 mm², or between 300 mm² and 2,000 mm², or between 400 mm² and 1,500 mm². Described another way, the total apertured area of the second zone 35b may preferably be between 30% and 60%, or between 35% and 55%, or between 40% and 50% of the total area of the second zone 35b.

As can be understood, an important aspect of the webs 33 of the present disclosure is the different features of the first zone and the second zone in order for the zones to efficiently handle different bodily exudates. The above description has described possible features of each of the zones which may be used together in any combination. The following section describes specific example webs according to aspects of the present disclosure which have been found useful in efficiently handling both urine and BM exudates by having differentiated first and second zones.

### First Example Materials

First example materials of the present disclosure were formed to have the variety of combinations of features as described below. These first example materials are two-layer laminate webs comprising a first upper web and a second lower web. The first upper web and the second lower web, for example precursor webs 133a, 133b as described above, were combined through an embossing and aperturing process including passing through a first nip with a first roll of the nip comprising protrusions for forming embossments and apertures and a second roll of the nip comprising corresponding recesses for receiving the embossing and aperturing protrusions of the first roll.

Through this process, a first visually distinct zone and a second visually distinct zone were imparted onto the first example materials. The first visually distinct zone and the second visually distinct zone are individually contiguous and contain morphological features with a boundary defined between the zones where the morphological features are present and other portions of the first example materials where the morphological of the zones are not present. For example, in such other portions either no morphological features may be present or morphological features different from those of the zones are present.

The first zones of the first example materials are configured to efficiently handle BM exudates. For this purpose, the first zones desirably are configured to have a total area of between 4,000 mm² and 8,000 mm² and to comprise embossments, apertures, and protrusions. In these first example materials, the apertures are disposed within the embossments, and the apertures are located at distal ends of protrusions which extend from the embossments. The embossments are located at least partially within the first upper web, beginning at a first surface of the first example materials and extending toward the second surface of the first example materials. The total embossed area is between 70% and 95% of the total area encompassed by the first zone in different first example materials. The embossments have a depth of between 50% and 70% of an overall thickness in different first example materials, with the thickness of different first example materials ranging from 1.5 mm to 3.5 mm.

The protrusions extend from within the embossments toward the second surface of the first example materials and protrude beyond the second surface. Distal ends of the protrusions define apertures which have a circle shape or oval shape. Desirably, the areas defined by the apertures of the first zone of the first example materials are between 0.5 mm² and 8 mm² or between 0.5 mm² and 4 mm². The first zone preferably has a total area defined by the apertures of between 90 mm² and 2,000 mm². Although, where the first example materials have a smaller area, the total area defined by the apertures may be between 90 mm² and 1,000 mm².

The second zone is configured to efficiently handle urine exudates. For this purpose, the second zone is configured to have a total area of between 3,750 mm² and 5,500 mm² and to comprise protrusions and apertures. The protrusions extend from the second surface of the first example materials, as do the protrusions of the first zone. Distal ends of the protrusion define apertures which may have a different shape than the apertures of the first zone. For example, the apertures of the second zone may be circle shaped where the apertures of the first zone are oval shaped or may be oval shaped where the apertures of the second zone are circle shaped. Alternatively, the apertures of the second zone may be square shaped or rectangular shaped.

The areas defined by the apertures of the second zone are less than the areas defined by apertures of the first zone. Desirably, the areas defined by the apertures of the second zone are between 0.75 mm² and 3.0 mm². The second zone preferably has a total area defined by the apertures of between 400 mm² and 1,500 mm².

### Second Example Materials

Second example materials of the present disclosure were formed to have the variety of combinations of features as described below. These second example materials are similar two-layer laminate webs as the first example materials and may be formed in a similar manner. As well, the second example materials have a first visually distinct zone and a second visually distinct zone imparted onto the second example materials.

The first zone is configured to efficiently handle BM exudates. For this purpose, the first zone desirably is configured to have a total area of between 4,000 mm² and 8,000 mm² and to comprise embossments, apertures, and protrusions. In the second example materials, the apertures are disposed within the embossments, and the apertures are located at distal ends of protrusions which extend from the embossments. The embossments are located at least partially within the first upper web, beginning at a first surface of the second example materials and extending toward the second surface of the second example materials. The total embossed area comprises between 70% and 95% of the total area encompassed by the first zone. The embossments have a depth of between 50% and 70% of an overall thickness of the second example materials, with the thickness of the different second example materials ranging from 1.5 mm to 3.5 mm.

The protrusions extend from within the embossments toward the second surface of the second example materials and protrude beyond the second surface. The protrusions extend beyond the second surface of the second example materials for a distance between 0.5 mm and 1.5 mm. Distal ends of the protrusions define apertures which have a circle shape or oval shape. Desirably, the areas defined by the apertures are between 0.5 mm² and 8 mm² or between 0.5 mm² and 4 mm². The first zone preferably has a total area defined by the apertures of between 90 mm² and 2,000 mm². Although, where the different example materials have a smaller area, the total area defined by the apertures may be between 90 mm² and 1,000 mm².

The second zone is configured to efficiently handle urine exudates. For this purpose, the second zone is configured to have a total area of between 3,750 mm² and 5,500 mm² and to comprise protrusions and apertures. The protrusions extend from the second surface of the second example materials, as do the protrusions of the first zone. Preferably, the protrusions of the second zone protrude outwardly from the second surface of the second example materials for a distance of between 0.1 mm and 0.5 mm. Distal ends of the protrusion define apertures which may have a different shape than the apertures of the first zone. For example, the apertures of the second zone may be circle shaped where the apertures of the first zone are oval shaped or may be oval shaped where the apertures of the second zone are circle shaped. Alternatively, the apertures of the second zone may be square shaped or rectangular shaped.

The areas defined by the apertures of the second zone are less than the areas defined by apertures of the first zone. Desirably, the areas defined by the apertures of the second zone are between 0.75 mm² and 3.0 mm². The second zone preferably has a total area defined by the apertures of between 400 mm² and 1,500 mm².

### Third Example Materials

Third example materials of the present disclosure were formed to have the variety of combinations of features as described below. These third example materials are similar two-layer laminate webs as the first example materials and may be formed in a similar manner. As well, the third example materials have a first visually distinct zone and a second visually distinct zone imparted onto the third example materials.

The first zone is configured to efficiently handle BM exudates. For this purpose, the first zone desirably is configured to have a total area of between 4,000 mm² and 8,000 mm² and to comprise embossments, apertures, and protrusions. In the third example materials, the apertures are disposed within the embossments, and the apertures are located at distal ends of protrusions which extend from the embossments. The embossments are located at least partially within the first upper web, beginning at a first surface of the third example materials and extending toward the second surface of the third example materials. The total embossed area should comprise between 70% and 95% of the total area encompassed by the first zone. The embossments preferably have a depth of between 50% and 70% of an overall thickness of the example material 3, with the thickness of the third example materials ranging from 1.5 mm to 3.5 mm.

The protrusions extend from within the embossments toward the second surface of the third example materials and protrude beyond the second surface. Distal ends of the protrusions define apertures which have a circle shape or oval shape.

The protrusions have a proximal base and a distal end, with the distal end protruding outward from the second surface of the third example materials. In the third example materials, the proximal base of the protrusions is disposed closer to the first surface of the third example materials than is a floor of the embossment. That is, the location of the proximal base of the protrusions is not at the bottom of the embossment. As described previously, this may advantageously form pockets within the embossments between the wall of the embossments and the proximal base of the protrusions where BM exudates can be temporarily stored.

### Fourth Example Materials

Fourth example materials of the present disclosure were formed to have the variety of combinations of features as described below. These fourth example materials are similar two-layer laminate webs as the first example materials and may be formed in a similar manner. As well, the fourth example materials have a first visually distinct zone and a second visually distinct zone imparted onto the fourth example materials.

The first zone is configured to efficiently handle BM exudates. For this purpose, the first zone desirably is configured to have a total area of between 4,000 mm² and 8,000 mm² and to comprise embossments, apertures, and protrusions. In these fourth example materials, the apertures are disposed within the embossments, and the apertures are located at distal ends of protrusions which extend from the embossments. The embossments are located at least partially within the first upper web, beginning at a first surface of the fourth example materials and extending toward the second surface of the fourth example materials. The total embossed area should comprise between 70% and 95% of the total area encompassed by the first zone. The embossments preferably have a depth of between 50% and 70% of an overall thickness of the fourth example materials, with the thickness of different example materials ranging from 1.5 mm to 3.5 mm.

The embossments of the first zone have an opening with an opening width, a minimum interior width, and a maximum interior width. The portion of an embossment having the minimum interior width is positioned to be between the opening and the portion of the embossment with maximum interior width.

The protrusions extend from within the embossments toward the second surface of the fourth example materials and protrude beyond the second surface. Distal ends of the protrusions define apertures which have a circle shape or oval shape.

The protrusions have a proximal base and a distal end, with the distal end protruding outward from the second surface of the fourth example materials. In the fourth example materials, the proximal base of the protrusions is disposed closer to the first surface of the fourth example materials than the portion of the embossment having the maximum interior width. The proximal base of the protrusions is also positioned such that the portion of the embossment having the minimum interior width is disposed closer to the first surface of the fourth example materials than the proximal base of the protrusion. Such a configuration may advantageously form pockets within the embossments between the wall of the embossments and the proximal base of the protrusions where BM exudates can be temporarily stored. Such pockets are covered by overhangs of the embossments, thereby preventing their direct contact with a wearer's skin.

The second zone of fourth example materials are configured to efficiently handle urine exudates. For this purpose, the second zone is configured to have a total area of between 3,750 mm² and 5,500 mm² and to comprise protrusions and apertures. The protrusions extend from the second surface of the fourth example materials, as do the protrusions of the first zone. Preferably, the protrusions of the second zone protrude outwardly from the second surface of the fourth example materials for a distance of between 0.1 mm and 0.5 mm. Distal ends of the protrusion define apertures which may have a different shape than the apertures of the first zone. For example, the apertures of the second zone may be circle shaped where the apertures of the first zone are oval shaped or may be oval shaped where the apertures of the second zone are circle shaped. Alternatively, the apertures of the second zone may be square shaped or rectangular shaped.

The areas defined by the apertures of the second zone are less than the areas defined by apertures of the first zone. Desirably, the areas defined by the apertures of the second zone are between 0.75 mm² and 3.0 mm². The second zone preferably has a total area defined by the apertures of between 400 mm² and 1,500 mm².

All of these first through fourth example materials can be combined with an absorbent article chassis such that the second lower webs are coupled with the absorbent article chassis. In this manner, the protrusions of the first zones of the first through fourth example materials protrude from the second lower web toward the backsheet of such absorbent articles. Such absorbent articles containing any of these first through fourth example materials produced good results in limiting BM exudate spread, BM exudate update, urine exudate uptake, and urine exudate rewet performance as compared to control materials and articles.

As described with respect to the specific first through fourth example materials, webs 33 of the present disclosure may be two-layer laminate webs comprising a first upper web and a second lower web. The first upper web and the second lower web can be combined through an embossing and aperturing process including passing through a first nip with a first roll of the nip comprising protrusions for forming embossments and apertures and a second roll of the nip comprising corresponding recesses for receiving the embossing and aperturing protrusions of the first roll.

When combining the first upper web and a second lower web, it is important to ensure sufficient lamination strength throughout the web 33. In order to effectively achieve sufficient lamination strength throughout webs 33, it may be desirable to impart lamination features to the web 33 as part of the embossing and aperturing process. Exemplary lamination features of the present disclosure can be seen with respect to lamination features 191 of FIGS. 7 and 8. Such lamination features 191 may comprise embossments, apertures, or both. In at least some embodiments, the lamination features 191 preferably have apertures 195 surrounded by embossments 193.

The lamination features 191 can be disposed in a staggered manner throughout the web 33 such that lamination features 191 of a first row of lamination features 191 extending in the transverse direction 32 and a second, longitudinally adjacent row of lamination features 191 extending in the transverse direction 32 do not have any transverse overlap. Alternatively, such lamination features 191 of longitudinally adjacent rows have a transverse overlap of less than 25% of a width of the lamination features 191. When forming web 33, the web 33 may be cut to form the longitudinally extending side edges 115, 117. Staggering the lamination features 191 helps to ensure that regardless of where the exact cut is to form the web 33, there are enough lamination features 191 present proximate the longitudinally extending side edges 115, 117 to prevent long unbonded segments, as can happen where the lamination features 191 are in alignment, or have transverse overlaps of greater than 25%, depending on exact location of cuts of the web 33.

Further, it may be important for registration purposes that the lamination features 191 are disposed away from the second zone a given distance. For example, some useful registration processes for registering web 33 within an absorbent article chassis include visual registration techniques identifying apertures within the second zone 35b of the web 33. Accordingly, placement of the lamination features 191 too close to the second zone 35b can interfere with such registration processes. In embodiments according to the present disclosure, the lamination features 191 are preferably disposed at least 5 mm, at least 7.5 mm, or at least 10 mm away from transversely extending front and rear edges of the second zone 35b.

In order to ensure sufficient lamination strength between the first upper web and a second lower web, such lamination features 191 may cover between 25% and 45% of an area of the web 33 outside of the first zone and the second zone 35a, 35b. Such areas are defined by addition of the individual embossments and/or apertures forming the lamination features 191. The imparted morphological features of the first zone and the second zone 35a, 35b provide sufficient lamination strength within those zones.

Suitable materials for such two-layer webs 33 include where the first upper web has a basis weight of between 10 gsm and 40 gsm, and more preferably between 17.5 gsm and 32.5 gsm. The first upper web can be a spunbonded web, including spunbond laminates such as a spunbond/meltblown/spunbond (“SMS”) web or the like, or a bonded-carded-web composed of natural fibers, synthetic fibers or combinations thereof. In other embodiments, the first upper web is preferably a through-air bonded carded web (TABCW), but could otherwise be any of a thermally bonded carded web, a wet-laid web, a coform web, or a hydraulically entangled web. The web may comprise fibers comprised of polyethylene (PE) fibers or both PET and polyethylene terephthalate (PET) polymers. In certain embodiments, the first upper web is formed of a blend of a first group of fibers and a second group of fibers. The first group of fibers may have deniers between 1.7 and 2.5 or between 1.8 and 2.2, while the second group of fibers have deniers of between 1.0 and 2.0, or between 1.25 and 1.75. The first group of fibers may be less hydrophilic than the second group of fibers.

The second lower web may have a basis weight of between 20 gsm and 45 gsm, and more preferably between 25 gsm and 40 gsm. The second lower web can, similarly to the first upper web, be a spunbonded web, including spunbond laminates such as a spunbond/meltblown/spunbond (“SMS”) web or the like, or a bonded-carded-web composed of natural fibers, synthetic fibers or combinations thereof. In other embodiments, the second lower web is preferably a through-air bonded carded web (TABCW), but could otherwise be any of a thermally bonded carded web, a wet-laid web, a coform web, or a hydraulically entangled web. The web may comprise fibers comprised of polyethylene (PE) fibers or both PET and polyethylene terephthalate (PET) polymers. In certain embodiments, the second lower web is formed of a single type of fibers having deniers between 3 and 5 or between 3.5 and 4.5 and is hydrophobic.

As described throughout, the exemplary web 33 of the present disclosure have been described as topical liner materials which can be coupled to a bodyfacing surface of an absorbent article (e.g. a body facing liner). However, it should be understood that such webs 33 do not need to be separate topical liners applied to a bodyside liner. Instead, the features of the web 33 described herein can be formed into a bodyside liner such that there is no topical liner. Rather, the bodyside liner 28 itself may have such first zones and second zones configured differently to efficiently handle different BM and urine exudates.

When incorporated into an absorbent article 10, 210, the web 33 may be positioned such that the first zone 35a and the second zone 35b are disposed wholly inboard of the distal ends of the projection portions of the flaps 50, 52, as can best be seen in FIGS. 2, 4, and 12. Such positioning helps to maintain the visual effectiveness of the different regions in communicating the different functions of the different zones. As well, when incorporated into an article, the web 33 may be incorporated such that the protrusions 109 protruding from the second surface of the web 33 are facing inward within the articles 10, 210 such that they project toward the backsheet 26 of the article. In this manner, the second surface of the web 33 may sit slightly above the absorbent article chassis and provide void volume underneath the web 33. Such void volume can be particularly important for handling BM exudates and transferring such BM exudates into an interior of the article 10, 210 and away from a wearer's skin.

### Tested Material 1

A first tested material, labeled as tested material 1 herein, was produced to have features according to aspects of the present disclosure as represented by web 33 of FIGS. 7-11. The tested material 1 is a dual-layer laminate material formed through a combined embossing and aperturing process. The combined embossing and aperturing process included passing two separated web materials a first nip with a first roll of the nip comprising protrusions for forming embossments and apertures and a second roll of the nip comprising corresponding recesses for receiving the embossing and aperturing protrusions of the first roll. The combined embossing and aperturing process laminated the two separate webs into a single laminate material and formed two discrete but individually contiguous and visually distinct regions within the formed laminate material. These regions are termed a first zone and a second zone herein.

The first web material was a 25 gsm TABCW material comprised of a blend of 2.0 denier PE/PET fibers and 1.5 denier PE/PET fibers having different hydrophilicities. The second web material was a 33 gsm TABCW material comprised of 4.0 denier PE/PET fibers. Further, a construction adhesive was applied between the two materials prior to the embossing and aperturing process to result in a 1.6 gsm layer of adhesive. The laminate material was cut to be 90 mm in width and have a length of 206 mm.

The tested material 1 was formed such that the first zone has embossment, aperture, and protrusion morphological features while the second zone has aperture and protrusion morphological features. TABLE 1 below details various aspects of the tested material 1, in terms of the average aperture area of the apertures of the first zone and the second zone, as well as the total aperture area of the first zone and the second zone.

The various measurements detailed herein may be obtained using any accepted measurement techniques within the nonwoven industry. In particular, the measurements detailed here were obtained utilizing the following test set-up:

### Material Sample Analysis Test Set-Up

The Material Sample Analysis Test Set-Up as described herein can be used for determining various measured aspects of a sample material, including length, width, depth, and other distances, as well as areas. The following described set-up should be understood as only one exemplary suitable set-up for capturing the various measurements described herein. Other industry acceptable set-ups could be used, including substitution of various components for known equivalent components. One having ordinary skill in the art would understand where component substitutions or set-up variations would produce differing results as compared to the described set-up. Such differences should be avoided.

The set-up used for acquiring the images of the sample materials herein and to determine the different values detailed herein include included a CCD video camera (e.g., a Leica DFC 300 FX video camera available from Leica Microsystems of Heerbrugg, Switzerland) mounted on a standard support such as a Polaroid MP-4 Land Camera standard support formerly available from Polaroid Resource Center in Cambridge, Miss., and now potentially available from a resource such as eBay. The standard support was attached to a macro-viewer such as a KREONITE macro-viewer available from Dunning Photo Equipment, Inc., having an office in Bixby, Okla. An auto stage was placed on the upper surface of the macro-viewer. The auto stage was used to automatically move the position of a given sample for viewing by the camera. A suitable auto stage is Model H112, available from Prior Scientific Inc., having an office in Rockland, Mass.

The specimen or sample material was placed on the auto stage of a Leica Microsystems QWIN Pro Image Analysis system, under the optical axis of a 60 mm lens having an f-stop setting of 4, such as a Nikon AF Micro Nikkor, manufactured by Nikon Corporation, having an office in Tokyo, Japan. The lens was attached to the camera using a c-mount adaptor. The distance from the front face of the lens to the sample was approximately 55 cm. The sample was laid flat on the auto stage surface and any wrinkles removed by gentle stretching and/or fastening it to the auto stage surface using transparent adhesive tape at its outer edges. The sample surface was illuminated with incident fluorescent lighting provided by a 16 inch diameter, 40 watt, Circline fluorescent light, such as that manufactured by General Electric Company, having an office in Boston, Mass. The light was contained in a fixture that is positioned so it is centered over the sample and is approximately 3 cm above the sample surface. The illumination level of the light was controlled with a Variable Auto-transformer (not shown), type 3PN1010, available from Staco Energy Products Co. having an office in Dayton, Ohio. Transmitted light was also provided to the sample from beneath the auto stage by a bank of four, 2-foot, EMC, Double-End Powered LED tube lights which are dimmable and available from Fulight Optoelectronic Materials, LLC. The LED lights were covered with a diffusing plate. The diffusing plate was inset into, and forms a portion of, the upper surface of the macro-viewer. This illumination source was overlaid with black mask possessing a 3-inch by 3-inch opening. The opening was positioned so that it was centered under the optical axis of the camera and lens system. The distance from the fluorescent light opening to the surface of the auto stage was approximately 17 cm. The illumination level of the fluorescent light bank was also controlled with a separate power control box (not shown) configured for dimmable LED lights.

The image analysis software platform used to perform the measurements detailed herein was QWIN Pro (Version 3.5.1) available from Leica Microsystems, having an office in Heerbrugg, Switzerland. Although, other software, such as LAS Macro Editor, the next generation of software following QWIN Pro, could be used to perform the analysis. The system and images were also accurately calibrated using the QWIN software and a standard ruler with metric markings at least as small as one millimeter. The calibration is performed in the horizontal dimension of the video camera image. Units of millimeters per pixel are used for the calibration.

Thus, the method for measuring the various values described herein includes the step of performing measurements on the transmitted light image. Specifically, an image analysis tool is used to acquire and process images as well as perform measurements. Some measurements may be performed utilizing a program such as Quantimet User Interactive Programming System (QUIPS) language.

The below measurements of TABLE 1 and the further tables detailed herein, as well as the similar features described previously with respect to other embodiments of the present disclosure were all determined according to the Material Sample Analysis Test Set-Up described above. In order to determine average values, eight randomly selected features were captured and measured according to the above-described set-up and analysis and the average calculated. The average area values were calculated based on known formulas for determining areas from the determined average values of the features (e.g. lengths, widths, radiuses, etc.). However, other methods could be employed, such as utilizing the image analysis tool or program to automatically calculate, or calculate based on user input, direct area values and then averaging the eight separate direct area values.

TABLE 1 includes an average aperture length value, which is an aspect of the aperture morphological features 105 of the first and second zones 35a, 35b which extends in the longitudinal direction 30. The average aperture width value is a further aspect of the aperture morphological features 105 of the first and second zones 35a, 35b which extends in the transverse direction 32. The calculated average aperture area values were determined utilizing the determined lengths and widths along with an equation for calculating an area of an ellipse or a rectangle, which are the shapes of the aperture morphological features 105 of the first and second zones 35a, 35b, respectively. It should be understood that different formulas or methods could be employed where appropriate to determine areas where the aperture morphological features 105 are differently shaped, including utilizing the assistance of an analytical computer program to determine areas directly from the images captured according to the Material Sample Analysis Test Set-Up. The total number of aperture morphological features 105 was multiplied by the calculated average aperture areas to arrive at the total apertured area of the first and second zones 35a, 35b.

TABLE 1

| | | | | |
|---|---|---|---|---|
| Average | Average | | | |
| Aperture | Aperture | Average | | Total |
| Length (mm) | Width (mm) | Aperture | Number of | Aperture |
| [longi- | [trans- | Area | Apertures | Area |
| tudinal] | verse] | (mm\hat{ }2) | (#) | (mm\hat{ }2) |

First Zone

| | | | | | |
|---|---|---|---|---|---|
| Tested | 1.0 | 1.0 | 1.00 | 465 | 465 |
| Material | | | | apertures | |
| 1 | | | | | |

Second Zone

| | | | | | |
|---|---|---|---|---|---|
| Tested | 2.58 | 1.38 | 2.80 | 126 | 358.59 |
| Material | | | | apertures | |
| 1 | | | | | |

TABLE 2 below details further aspects of the tested material 1, including the total apertured area of the first zone 35a, the second zone 35b, and a total apertured area of the material. The % aperture areas of the first/second zones 35a, 35b are calculated as a function of the total area of the material. For example, the total aperture area of the second zone 35b is 465 mm², which results in a percentage of the total material area of 465 divided by 18,450, which equals approximately 2.5%. The total aperture area of the first/second zones 35a, 35b were determined by multiplying the determined average aperture areas of the given zones and multiplied by the number of apertures in the same zone

TABLE 2

| | | Total Average | Total Average | % Aperture | | Total % |
|---|---|---|---|---|---|---|
| | Total Area | Aperture Area | Aperture Area | Area | % Aperture | Aperture |
| | of Material | Second Zone | First Zone | Second | Area First | Area of |
| | (mm\hat{ }2) | (mm\hat{ }2) | (mm\hat{ }2) | Zone | Zone | Material |
| Tested Material 1 | 18,540 | 465 | 358.59 | 2.5% | 1.9% | 4.4% |

TABLE 3 below details aspects of the embossment morphological features 101 of the first zone. The embossment morphological features 101 of the tested material 1 are generally diamond shaped. Accordingly, the calculated areas were done utilizing an equation for an area of a diamond from measured average maximum width and length values of individual embossment morphological features 101. As described above, different formulas or methods could be employed where appropriate to determine areas where the embossment morphological features 101 are differently shaped. The total number of embossment morphological features 101 was multiplied by the calculated average embossed area to arrive at the total embossed area of the first zone. Finally, the % embossed area details the total percentage of the material area which is embossed. This is calculated as dividing the total embossed area by the total material area (from TABLE 2).

TABLE 3
First Zone Embossed Area

| | Average | Average | | | Total | |
|---|---|---|---|---|---|---|
| | Embossed | Embossed | Average | Number of | Embossed | % |
| | Length (mm) | Width (mm) | Embossed | Embossed | Area | Embossed |
| | [longitudinal] | [transverse] | Area (mm\hat{ }2) | Features (#) | (mm\hat{ }2) | Area |
| Tested Material 1 | 11.32 | 5.37 | 30.43 | 126 features | 3833.61 | 20.7% |

TABLE 4 below details features of the spacings between the embossment morphological features 101 for the first zone. For instance, the minimum width between embossment features measurement details the width between closest adjacent embossment morphological features 101 as shown by reference number 167 in FIG. 9. The longitudinal length between embossment features measurement details a distance in the longitudinal direction between embossment morphological features 101 disposed in a same longitudinally extending column. Finally, the transverse length between embossment features measurement details a distance in the transverse direction between embossment morphological features 101 disposed in a same transverse row, as depicted by reference number 169 in FIG. 9. Similar to the other measurements detailed herein, the values for eight randomly selected features were measured and then an average value was calculated based on the measurements.

TABLE 4
First Zone Embossment Morphological
Features 101 Relative Positioning

| | Minimum Width | Longitudinal | Transverse |
|---|---|---|---|
| | Between | Length Between | Length Between |
| | Embossment | Embossment | Embossment |
| | Features (mm) | Features (mm) | Features (mm) |
| Tested | 1.87 | 4.20 | 3.98 |
| Material 1 | | | |

TABLE 5 below details further aspects of the embossment morphological features 101 and the protrusion morphological features 159 of the tested material 1. To measure the described features, eight samples of the tested material 1 were cut in the transverse direction. Example appropriate cutting methods include utilizing a razor-sharp blade, lasers, or high-pressure water jets. No matter the methodology, care should be taken such that the transverse cut extends directly through the center of at least one aperture morphological feature 105 and that the cut had minimal impact on the structure of the material. The material thickness adjacent to the embossment morphological feature was measured for each sample, as indicated by reference number 143 of FIG. 10. A maximum depth of the embossment morphological feature 101 was further determined for each sample, as indicated by reference number 141. Further, a height for which the protrusion morphological feature 159 extends beyond the second surface 128 of the tested material 1 was determined for each sample, as indicated by reference number 168. These measured values were then used to calculated average values. The average material thickness and the average protrusion height values were added to result in an average overall material thickness parameter relevant for the portions of the tested material 1 containing the protrusion morphological features 159. The % thickness value details the additional thickness provided to the tested material 1 by the protrusion morphological feature 159.

TABLE 5
First Zone Embossment 101 and Protrusion
Morphological Feature 159 Measurements

| Average | Average | | Average | Average | |
|---|---|---|---|---|---|
| Material | Embossed | % | Protrusion | Overall | % |
| Thickness | Depth | Thickness | Height | Thickness | Thickness |
| (mm) | (mm) | Embossed | (mm) | (mm) | Increase |
| 2.44 | 1.61 | 66.15% | 0.78 | 3.22 | 32.0% |

TABLE 6 below details further aspects of the embossment morphological features 101 of the first zone. The average embossment opening width value details the width of the embossment at the first surface of the tested material 1. This feature is indicated by reference number 171 of FIG. 10. The embossment width minimum value details the minimum width of the embossment defined by the walls of the embossment through the depth of the embossment. This feature is indicated by reference number 173 of FIG. 10. The embossment width maximum value details the maximum width of the embossment defined by the walls of the embossment through the depth of the embossment. This feature is indicated by reference number 175 of FIG. 10. The % width increase value details how much larger the maximum width of the embossment is than the minimum width of the embossment.

TABLE 6
First Zone Embossed Morphological Feature 101 Measurements

| Average | Average | Embossment | Embossment | |
|---|---|---|---|---|
| Embossed | Embossment | Width | Width | |
| Depth | Opening Width | Minimum | Maximum | % Width |
| (mm) | (mm) | (mm) | (mm) | Increase |
| 1.61 | 5.37 | 5.11 | 5.9 | 15.5% |

TABLE 7 below details further aspects of the protrusion morphological features 159 of the first zone. The average protrusion opening width value details the width of the protrusion morphological feature 159 at its proximal base 162. This feature is indicated by reference number 171 of FIG. 10. The average aperture width value details the width of the aperture morphological features 105 defined by the distal end 164 of the protrusion morphological features 159. This feature is indicated by reference number 176 of FIG. 10. The % width reduction value details the amount by which the opening width has been reduced between the proximal base 162 and the distal end 164 of the protrusion morphological feature 159. This feature is indicated by reference number 175 of FIG. 10. The average protrusion height value details the amount by which the protrusion morphological feature 159 extends beyond the second surface 128 of the tested material 1. This feature is indicated by reference number 168 of FIG. 10.

TABLE 7
First Zone Protrusion Morphological Feature 159 Measurements

| Average | | | |
|---|---|---|---|
| Protrusion | Average | | Average |
| Opening Width | Aperture | % Width | Protrusion |
| (mm) | Width (mm) | Reduction | Height (mm) |
| 2.75 | 1.12 | 59.3% | 0.78 |

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EMBODIMENTS

Embodiment 1: An absorbent article extending in a longitudinal direction and a transverse direction and comprising: a bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct; a backsheet material; an absorbent body disposed between the bodyside liner material and the backsheet material; wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more protrusion and/or one or more aperture morphological features, and wherein the morphological features of each of the first zone and the second zone define outer boundaries of the first zone and the second zone, the outer boundaries of the first zone forming a first shape and the outer boundaries of the second zone forming a second shape that is different from the first shape.

Embodiment 2: The absorbent article of embodiment 1, wherein the shapes defined by the outer boundaries of the first zone and the second zone comprise geometric shapes.

Embodiment 3: The absorbent article of any one of embodiments 1-2, wherein the shape defined by the outer boundary of the second zone is a square and shape defined by the outer boundary of the first zone is a polygon have a number of sides greater than 4.

Embodiment 4: The absorbent article of any one of embodiments 1-3, wherein an average area defined by the individual aperture morphological features of the first zone is greater than an average area defined by the individual aperture morphological features of the second zone.

Embodiment 5: The absorbent article of embodiment 4, wherein a total area defined by the aperture morphological features of the first zone is greater than a total area defined by the aperture morphological features of the second zone.

Embodiment 6: The absorbent article of any one of embodiments 1-5, wherein a total area defined by the aperture morphological features of the first zone is greater than a total area defined by the aperture morphological features of the second zone.

Embodiment 7: The absorbent article of any one of embodiments 1-6, wherein the protrusion morphological features of the second zone protrude away from the second garment facing surface for a distance that is less than a distance the protrusion morphological features of the first zone protrude away from the second garment facing surface.

Embodiment 8: The absorbent article of any one of embodiments 1-7, wherein the second zone does not comprise embossment morphological features.

Embodiment 9: The absorbent article of any one of embodiments 1-8, wherein the protrusion morphological features of the first zone have proximal ends and distal ends, the embossment morphological features of the first zone have an opening at the first body facing surface comprising an opening width, a portion having an interior minimum width, and a portion having an interior maximum width, and the protrusion morphological features are disposed within the embossment morphological features such that the proximal ends of the protrusion morphological features are disposed away from the opening a distance that is less than a distance between the opening and the portion of the embossment having the embossment interior maximum width and greater than a distance between the opening and the portion of the embossment having the embossment interior minimum width.

Embodiment 10: The absorbent article of any one of embodiments 1-9, wherein each of the first zone and the second zone are disposed between 7.5 mm and 20 mm from longitudinally extending side edges of the bodyside liner material.

Embodiment 11: The absorbent article of any one of embodiments 1-10, wherein the first zone is spaced longitudinally from the second zone between 5 mm and 30 mm.

Embodiment 12: The absorbent article of any one of embodiments 1-11, wherein an area of the first zone is greater than an area of the second zone.

Embodiment 13: The absorbent article of any one of embodiments 1-12, wherein an area of the first zone is between 4,000 mm and 8,000 mm and an area of the second zone is between 3,000 mm and 4,500 mm.

Embodiment 14: The absorbent article of any one of embodiments 1-13, wherein a total area defined by the aperture morphological features of the first zone is between 2.5% and 14% of a total area of the first zone, and wherein the second zone comprises aperture morphological features with a total area defined by the aperture morphological features of the second zone is between 35% and 55% of a total area of the second zone.

Embodiment 15: The absorbent article of any one of embodiments 1-14, wherein a total area defined by the embossment morphological features of the first zone is between 65% and 95% of a total area of the first zone.

Embodiment 16: The absorbent article of any one of embodiments 1-15, wherein a total area defined by the aperture morphological features of the first zone is between 2.5% and 14% of a total area of the first zone.

Embodiment 17: The absorbent article of any one of embodiments 1-16, wherein an average area defined by the aperture morphological features of the first zone is between 10% and 40% of an average area defined by the embossment morphological features of the first zone.

Embodiment 18: The absorbent article of any one of embodiments 1-17, wherein the protrusion morphological features comprise proximal ends and distal ends with the distal ends defining the aperture morphological, and wherein the protrusion morphological features are funnel shaped such that openings defined by the proximal ends of the protrusion morphological features have greater areas than the aperture morphological features.

Embodiment 19: The absorbent article of any one of embodiments 1-18, further comprising a secondary liner material having a body facing surface and a garment facing surface, and wherein the bodyside liner material is a topical liner material with the garment facing surface of the bodyside liner material coupled to the body facing surface of the secondary liner material.

Embodiment 20: An absorbent article extending in a longitudinal direction and a transverse direction and comprising: bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct; a backsheet material; an absorbent body disposed between the bodyside liner material and the backsheet material; wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more aperture morphological features, and wherein an average area of the aperture morphological features of the first zone is larger than the an average area of the aperture morphological features of the second zone, and wherein a total area of the aperture morphological features of the first zone is larger than a total area of the aperture morphological features of the second zone.

Embodiment 21: The absorbent article of embodiment 20, wherein the second zone comprises aperture morphological features, wherein boundaries of the aperture morphological features of the first zone define a first shape, boundaries of the aperture morphological features of the second zone define a second shape, with the first shape being different than the second shape.

Embodiment 22: The absorbent article of any one of embodiments 20-21, wherein the second zone comprises aperture morphological features, wherein boundaries of the aperture morphological features of the first zone define a circle shape or an oval shape and boundaries of the aperture morphological features of the second zone define a square shape or a rectangle shape.

Embodiment 23: The absorbent article of any one of embodiments 20-22, wherein the protrusion morphological features of the second zone protrude away from the second garment facing surface for a distance that is less than a distance the protrusion morphological features of the first zone protrude away from the second garment facing surface.

Embodiment 24: The absorbent article of any one of embodiments 20-23, wherein the second zone does not comprise an embossment morphological feature.

Embodiment 25: The absorbent article of any one of embodiments 20-24, wherein the protrusion morphological features of the first zone have proximal ends and distal ends, the embossment morphological features of the first zone have an opening at the first body facing surface comprising an opening width, a portion having an interior minimum width, and a portion having an interior maximum width, and the protrusion morphological features are disposed within the embossment morphological features such that the proximal ends of the protrusion morphological features are disposed away from the opening a distance that is less than a distance between the opening and the portion of the embossment having the embossment interior maximum width and greater than a distance between the opening and the portion of the embossment having the embossment interior minimum width.

Embodiment 26: The absorbent article of any one of embodiments 20-25, wherein each of the first zone and the second zone are disposed between 7.5 mm and 20 mm from longitudinally extending side edges of the bodyside liner material.

Embodiment 27: The absorbent article of any one of embodiments 20-26, wherein the first zone is spaced longitudinally from the second zone between 5 mm and 30 mm.

Embodiment 28: The absorbent article of any one of embodiments 20-27, wherein an area of the first zone is greater than an area of the second zone.

Embodiment 29: The absorbent article of any one of embodiments 20-28, wherein an area of the first zone is between 4,000 mm and 8,000 mm and an area of the second zone is between 3,000 mm and 5,000 mm.

Embodiment 30: The absorbent article of any one of embodiments 20-29, wherein a total area defined by the aperture morphological features of the first zone is between 2.5% and 14% of a total area of the first zone, and wherein a total area defined by the aperture morphological features of the second zone is between 35% and 55% of a total area of the second zone.

Embodiment 31: The absorbent article of any one of embodiments 20-30, wherein a total area defined by the embossment morphological features of the first zone is between 65% and 95% of a total area of the first zone.

Embodiment 32: The absorbent article of any one of embodiments 20-31, wherein a total area defined by the aperture morphological features of the first zone is between 2.5% and 14% of a total area of the first zone.

Embodiment 33: The absorbent article of any one of embodiments 20-32, wherein an average area defined by the aperture morphological features of the first zone is between 10% and 40% of an average area defined by the embossment morphological features of the first zone.

Embodiment 34: The absorbent article of any one of embodiments 30-33, wherein the protrusion morphological features comprise proximal ends and distal ends with the distal ends defining the aperture morphological, and wherein the protrusion morphological features are funnel shaped such that openings defined by the proximal ends of the protrusion morphological features have greater areas than the aperture morphological features.

Embodiment 35: The absorbent article of any one of embodiments 20-34, further comprising a secondary liner material having a body facing surface and a garment facing surface, and wherein the bodyside liner material is a topical liner material with the garment facing surface of the bodyside liner material coupled to the body facing surface of the secondary liner material.

Embodiment 36: An absorbent article extending in a longitudinal direction and a transverse direction and comprising: a bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct; a backsheet material; and an absorbent body disposed between the bodyside liner material and the backsheet material; wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more aperture morphological features, and wherein an average area of the aperture morphological features of the first zone is larger than an average area of the aperture morphological features of the second zone and wherein boundaries of the aperture morphological features of the first zone form a first shape and boundaries of the aperture morphological features of the second zone form a second shape that is different than the first shape.

Embodiment 37: The absorbent article of embodiment 36, wherein boundaries of the aperture morphological features of the first zone define a circle shape or an oval shape and boundaries of the aperture morphological features of the second zone define a square shape or a rectangle shape.

Embodiment 38: The absorbent article of any one of embodiments 36-37, wherein the protrusion morphological features of the second zone protrude away from the second garment facing surface for a distance that is less than a distance the protrusion morphological features of the first zone protrude away from the second garment facing surface.

Embodiment 39: The absorbent article of any one of embodiments 36-38, wherein the second zone does not comprise an embossment morphological feature.

Embodiment 40: The absorbent article of any one of embodiments 36-39, wherein the protrusion morphological features of the first zone have proximal ends and distal ends, the embossment morphological features of the first zone have an opening at the first body facing surface comprising an opening width, a portion having an interior minimum width, and a portion having an interior maximum width, and the protrusion morphological features are disposed within the embossment morphological features such that the proximal ends of the protrusion morphological features are disposed away from the opening a distance that is less than a distance between the opening and the portion of the embossment having the embossment interior maximum width and greater than a distance between the opening and the portion of the embossment having the embossment interior minimum width.

Embodiment 41: The absorbent article of any one of embodiments 36-40, wherein each of the first zone and the second zone are disposed between 7.5 mm and 20 mm from longitudinally extending side edges of the bodyside liner material.

Embodiment 42: The absorbent article of any one of embodiments 36-41, wherein the first zone is spaced longitudinally from the second zone between 5 mm and 30 mm.

Embodiment 43: The absorbent article of any one of embodiments 36-42, wherein an area of the first zone is greater than an area of the second zone.

Embodiment 44: The absorbent article of any one of embodiments 36-43, wherein an area of the first zone is between 4,000 mm and 8,000 mm and an area of the second zone is between 3,000 mm and 5,000 mm.

Embodiment 45: The absorbent article of any one of embodiments 36-44, wherein a total area defined by the aperture morphological features of the first zone is between 2.5% and 14% of a total area of the first zone, and wherein a total area defined by the aperture morphological features of the second zone is between 35% and 55% of a total area of the second zone.

Embodiment 46: The absorbent article of any one of embodiments 36-45, wherein a total area defined by the embossment morphological features of the first zone is between 65% and 95% of a total area of the first zone.

Embodiment 47: The absorbent article of any one of embodiments 36-46, wherein a total area defined by the aperture morphological features of the first zone is between 2.5% and 14% of a total area of the first zone.

Embodiment 48: The absorbent article of any one of embodiments 36-47, wherein an average area defined by the aperture morphological features of the first zone is between 10% and 40% of an average area defined by the embossment morphological features of the first zone.

Embodiment 49: The absorbent article of any one of embodiments 36-48, wherein the protrusion morphological features comprise proximal ends and distal ends with the distal ends defining the aperture morphological, and wherein the protrusion morphological features are funnel shaped such that openings defined by the proximal ends of the protrusion morphological features have greater areas than the aperture morphological features.

Embodiment 50: The absorbent article of any one of embodiments 36-49, further comprising a secondary liner material having a body facing surface and a garment facing surface, and wherein the bodyside liner material is a topical liner material with the garment facing surface of the bodyside liner material coupled to the body facing surface of the secondary liner material.

Embodiment 51: An absorbent article extending in a longitudinal direction and a transverse direction and comprising: a bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct; a backsheet material; and an absorbent body disposed between the bodyside liner material and the backsheet material; wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more aperture morphological features, and wherein an average area of the aperture morphological features of the first zone is larger than an average area of the aperture morphological features of the second zone and wherein an average height of the protrusion morphological features of the first zone protruding beyond the second garment facing surface is greater than an average height of the protrusion morphological features of the second zone protruding beyond the second garment facing surface.

Embodiment 52: The absorbent article of embodiment 51, wherein the shapes defined by the outer boundaries of the first zone and the second zone comprise geometric shapes.

Embodiment 53: The absorbent article of any one of embodiments 51-52, wherein the shape defined by the outer boundary of the second zone is a square and shape defined by the outer boundary of the first zone is a polygon have a number of sides greater than 4.

Embodiment 54: The absorbent article of any one of embodiments 51-53, wherein an average area defined by the aperture morphological features of the first zone is greater than an average area defined by the aperture morphological features of the second zone.

Embodiment 55: The absorbent article of any one of embodiments 51-54, wherein a total area defined by the aperture morphological features of the first zone is greater than a total area defined by the aperture morphological features of the second zone.

Embodiment 56: The absorbent article of any one of embodiments 51-55, wherein a total area defined by the aperture morphological features of the first zone is greater than a total area defined by the aperture morphological features of the second zone.

Embodiment 57: The absorbent article of any one of embodiments 51-56, wherein the protrusion morphological features of the second zone protrude away from the second garment facing surface for a distance that is less than a distance the protrusion morphological features of the first zone protrude away from the second garment facing surface.

Embodiment 58: The absorbent article of any one of embodiments 51-57, wherein the second zone does not comprise embossment morphological features.

Embodiment 59: The absorbent article of any one of embodiments 51-58, wherein the protrusion morphological features of the first zone have proximal ends and distal ends, the embossment morphological features of the first zone have an opening at the first body facing surface comprising an opening width, a portion having an interior minimum width, and a portion having an interior maximum width, and the protrusion morphological features are disposed within the embossment morphological features such that the proximal ends of the protrusion morphological features are disposed away from the opening a distance that is less than a distance between the opening and the portion of the embossment having the embossment interior maximum width and greater than a distance between the opening and the portion of the embossment having the embossment interior minimum width.

Embodiment 60: The absorbent article of any one of embodiments 51-59, wherein each of the first zone and the second zone are disposed between 7.5 mm and 20 mm from longitudinally extending side edges of the topical liner material.

Embodiment 61: The absorbent article of any one of embodiments 51-60, wherein the first zone is spaced longitudinally from the second zone between 5 mm and 30 mm.

Embodiment 62: The absorbent article of any one of embodiments 51-61, wherein an area of the first zone is greater than an area of the second zone.

Embodiment 63: The absorbent article of any one of embodiments 51-62, wherein an area of the first zone is between 4,000 mm and 8,000 mm and an area of the second zone is between 3,000 mm and 5,000 mm.

Embodiment 64: The absorbent article of any one of embodiments 51-63, wherein a total area defined by the aperture morphological features of the first zone is between 2.5% and 14% of a total area of the first zone, and wherein a total area defined by the aperture morphological features of the second zone is between 35% and 55% of a total area of the second zone.

Embodiment 65: The absorbent article of any one of embodiments 51-64, wherein a total area defined by the embossment morphological features of the first zone is between 65% and 95% of a total area of the first zone.

Embodiment 66: The absorbent article of any one of embodiments 51-65, wherein a total area defined by the aperture morphological features of the first zone is between 2.5% and 14% of a total area of the first zone.

Embodiment 67: The absorbent article of any one of embodiments 51-66, wherein an average area defined by the aperture morphological features of the first zone is between 10% and 40% of an average area defined by the embossment morphological features of the first zone.

Embodiment 68: The absorbent article of any one of embodiments 51-67, wherein the protrusion morphological features comprise proximal ends and distal ends with the distal ends defining the aperture morphological, and wherein the protrusion morphological features are funnel shaped such that openings defined by the proximal ends of the protrusion morphological features have greater areas than the aperture morphological features.

Embodiment 69: The absorbent article of any one of embodiments 51-68, further comprising a secondary liner material having a body facing surface and a garment facing surface, and wherein the bodyside liner material is a topical liner material with the garment facing surface of the bodyside liner material coupled to the body facing surface of the secondary liner material.

Embodiment 70: An absorbent article extending in a longitudinal direction and a transverse direction and comprising: a bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct; a backsheet material; and an absorbent body disposed between the bodyside liner material and the backsheet material; wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more protrusion and/or one or more aperture morphological features, and wherein areas of the aperture morphological features of the first zone are larger than areas of the aperture morphological features of the second zone, and wherein: the protrusion morphological features of the first zone have proximal ends and distal ends, the embossment morphological features of the first zone have an opening comprising an opening width, an interior minimum width, and an interior maximum width, and the protrusion morphological features are disposed within the embossment morphological features such that the proximal ends of the protrusion morphological features are disposed between the embossment interior maximum width and the embossment interior minimum width.

Embodiment 71: The absorbent article of embodiment 70, wherein the morphological features of each of the first zone and the second zones define outer boundaries of the first zone and the second zone, the outer boundaries of the first zone forming a first shape and the outer boundaries of the second zone forming a second shape that is different from the first shape.

Embodiment 72: The absorbent article of any one of embodiments 70-71, wherein the shapes defined by the outer boundaries of the first zone and the second zone comprise geometric shapes.

Embodiment 73: The absorbent article of any one of embodiments 70-73, wherein the shape defined by the outer boundary of the second zone is a square and shape defined by the outer boundary of the first zone is a polygon have a number of sides greater than 4.

Embodiment 74: The absorbent article of any one of embodiments 70-73, wherein the second zone comprises aperture morphological features, and wherein an average area defined by the aperture morphological features of the first zone are greater than an average area defined by the aperture morphological features of the second zone.

Embodiment 75: The absorbent article Embodiment 74, wherein the second zone comprises aperture morphological features, and wherein a total area defined by the aperture morphological features of the first zone is greater than a total area defined by the aperture morphological features of the second zone.

Embodiment 76: The absorbent article of any one of embodiments 70-75, wherein a total area defined by the aperture morphological features of the first zone is greater than a total area defined by the aperture morphological features of the second zone.

Embodiment 77: The absorbent article of any one of embodiments 70-76, wherein the protrusion morphological features of the second zone protrude for a distance away from the second garment facing surface that is less than a distance the protrusion morphological features of the first zone protrude away from the second garment facing surface.

Embodiment 78: The absorbent article of any one of embodiments 70-77, wherein the second zone does not comprise embossment morphological features.

Embodiment 79: The absorbent article of any one of embodiments 70-78, wherein the protrusion morphological features of the first zone have proximal ends and distal ends, the embossment morphological features of the first zone have an opening at the first body facing surface comprising an opening width, a portion having an interior minimum width, and a portion having an interior maximum width, and the protrusion morphological features are disposed within the embossment morphological features such that the proximal ends of the protrusion morphological features are disposed away from the opening a distance that is less than a distance between the opening and the portion of the embossment having the embossment interior maximum width and greater than a distance between the opening and the portion of the embossment having the embossment interior minimum width.

Embodiment 80: The absorbent article of any one of embodiments 70-79, wherein each of the first zone and the second zone are disposed between 7.5 mm and 20 mm from longitudinally extending side edges of the topical liner material.

Embodiment 81: The absorbent article of any one of embodiments 70-80, wherein the first zone is spaced longitudinally from the second zone between 5 mm and 30 mm.

Embodiment 82: The absorbent article of any one of embodiments 70-81, wherein an area of the first zone is greater than an area of the second zone.

Embodiment 83: The absorbent article of any one of embodiments 70-82, wherein an area of the first zone is between 4,000 mm and 8,000 mm and an area of the second zone is between 3,000 mm and 5,000 mm.

Embodiment 84: The absorbent article of any one of embodiments 70-83, wherein a total area defined by the aperture morphological features of the first zone is between 2.5% and 14% of a total area of the first zone, and wherein the second zone comprises aperture morphological features with a total area defined by the aperture morphological features of the second zone is between 35% and 55% of a total area of the second zone.

Embodiment 85: The absorbent article of any one of embodiments 70-84, wherein a total area defined by the embossment morphological features of the first zone is between 65% and 95% of a total area of the first zone.

Embodiment 86: The absorbent article of any one of embodiments 70-85, wherein a total area defined by the aperture morphological features of the first zone is between 2.5% and 14% of a total area of the first zone.

Embodiment 87: The absorbent article of any one of embodiments 70-86, wherein an average area defined by the aperture morphological features of the first zone is between 10% and 40% of an average area defined by the embossment morphological features of the first zone.

Embodiment 88: The absorbent article of any one of embodiments 70-87, wherein the protrusion morphological features comprise proximal ends and distal ends with the distal ends defining the aperture morphological, and wherein the protrusion morphological features are funnel shaped such that openings defined by the proximal ends of the protrusion morphological features have greater areas than the aperture morphological features.

Embodiment 89: The absorbent article of any one of embodiments 70-77, further comprising a secondary liner material having a body facing surface and a garment facing surface, and wherein the bodyside liner material is a topical liner material with the garment facing surface of the bodyside liner material coupled to the body facing surface of the secondary liner material.

Embodiment 90: An absorbent article extending in a longitudinal direction and a transverse direction and comprising: a bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct; a backsheet material; and an absorbent body disposed between the bodyside liner material and the backsheet material; wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material, wherein the second zone comprises one or more aperture morphological features, and wherein an average area of the aperture morphological features of the first zone is larger than an average area of the aperture morphological features of the second zone, and wherein: the embossment morphological features of the first zone have an opening comprising an opening width, an interior minimum width, and an interior maximum width, fibers of the first web form the opening of the embossment and the portion of the embossment forming the interior minimum width, and fibers of the first web and the second web being tangled together to form the portion of the embossment forming the interior maximum width.

Embodiment 91: The absorbent article of embodiment 90, wherein the morphological features of each of the first zone and the second zones define outer boundaries of the first zone and the second zone, the outer boundaries of the first zone forming a first shape and the outer boundaries of the second zone forming a second shape that is different from the first shape.

Embodiment 92: The absorbent article of any one of embodiments 90-91, wherein the shapes defined by the outer boundaries of the first zone and the second zone comprise geometric shapes.

Embodiment 93: The absorbent article of any one of embodiments 90-92, wherein the shape defined by the outer boundary of the second zone is a square and shape defined by the outer boundary of the first zone is a polygon have a number of sides greater than 4.

Embodiment 94: The absorbent article of any one of embodiments 90-93, wherein an average area defined by the aperture morphological features of the first zone are greater than an average area defined by the aperture morphological features of the second zone.

Embodiment 95: The absorbent article of embodiment 94, wherein a total area defined by the aperture morphological features of the first zone is greater than a total area defined by the aperture morphological features of the second zone.

Embodiment 96: The absorbent article of any one of embodiments 90-95, wherein a total area defined by the aperture morphological features of the first zone is greater than a total area defined by the aperture morphological features of the second zone.

Embodiment 97: The absorbent article of any one of embodiments 90-96, wherein the protrusion morphological features of the second zone protrude for a distance away from the second garment facing surface that is less than a distance the protrusion morphological features of the first zone protrude away from the second garment facing surface.

Embodiment 98: The absorbent article of any one of embodiments 90-97, wherein the second zone does not comprise embossment morphological features.

Embodiment 99: The absorbent article of any one of embodiments 90-98, wherein the protrusion morphological features of the first zone have proximal ends and distal ends, the embossment morphological features of the first zone have an opening at the first body facing surface comprising an opening width, a portion having an interior minimum width, and a portion having an interior maximum width, and the protrusion morphological features are disposed within the embossment morphological features such that the proximal ends of the protrusion morphological features are disposed away from the opening a distance that is less than a distance between the opening and the portion of the embossment having the embossment interior maximum width and greater than a distance between the opening and the portion of the embossment having the embossment interior minimum width.

Embodiment 100: The absorbent article of any one of embodiments 90-99, wherein each of the first zone and the second zone are disposed between 7.5 mm and 20 mm from longitudinally extending side edges of the topical liner material.

Embodiment 101: The absorbent article of any one of embodiments 90-100, wherein the first zone is spaced longitudinally from the second zone between 5 mm and 30 mm.

Embodiment 102: The absorbent article of any one of embodiments 90-101, wherein an area of the first zone is greater than an area of the second zone.

Embodiment 103: The absorbent article of any one of embodiments 90-102, wherein an area of the first zone is between 4,000 mm and 8,000 mm and an area of the second zone is between 3,000 mm and 5,000 mm.

Embodiment 104: The absorbent article of any one of embodiments 90-103, wherein a total area defined by the aperture morphological features of the first zone is between 2.5% and 14% of a total area of the first zone, and a total area defined by the aperture morphological features of the second zone is between 35% and 55% of a total area of the second zone.

Embodiment 105: The absorbent article of any one of embodiments 90-104, wherein a total area defined by the embossment morphological features of the first zone is between 65% and 95% of a total area of the first zone.

Embodiment 106: The absorbent article of any one of embodiments 90-105, wherein a total area defined by the aperture morphological features of the first zone is between 2.5% and 14% of a total area of the first zone.

Embodiment 107: The absorbent article of any one of embodiments 90-106, wherein an average area defined by the aperture morphological features of the first zone is between 10% and 40% of an average area defined by the embossment morphological features of the first zone.

Embodiment 108: The absorbent article of any one of embodiments 90-107, wherein the protrusion morphological features comprise proximal ends and distal ends with the distal ends defining the aperture morphological, and wherein the protrusion morphological features are funnel shaped such that openings defined by the proximal ends of the protrusion morphological features have greater areas than the aperture morphological features.

Embodiment 109: The absorbent article of any one of embodiments 90-108, further comprising a secondary liner material having a body facing surface and a garment facing surface, and wherein the bodyside liner material is a topical liner material with the garment facing surface of the bodyside liner material coupled to the body facing surface of the secondary liner material.

Embodiment 110: The absorbent article of any one of embodiments 1-109, wherein the embossment morphological features have a maximum depth from the first surface of the bodyside liner material, and wherein openings of the protrusion morphological features are disposed a distance away from the first surface that is less than the maximum depth of the embossment morphological features.

Embodiment 111: The absorbent article of any one of embodiments 1-110, wherein the protrusion morphological features are disposed within the embossment morphological features.

Embodiment 112: The absorbent article of any one of embodiments 1-111, wherein the distal ends of the protrusion morphological features of the first zone define the aperture morphological features of the first zone.

Embodiment 113: The absorbent article of any one of embodiments 1-112, wherein the second zone comprises one or more aperture morphological features.

Embodiment 114: The absorbent article of any one of embodiments 1-113, wherein the second zone comprises one or more protrusion morphological features.

Embodiment 115: The absorbent article of any one of embodiments 1-114, wherein the second zone comprises one or more protrusion morphological features having distal ends and wherein the distal ends of the protrusion morphological features of the second zone define the aperture morphological features of the second zone.

What is claimed:

1. An absorbent article extending in a longitudinal direction and a transverse direction comprises:
a bodyside liner material having a body facing surface and a garment facing surface;
a backsheet material;
an absorbent body disposed between the bodyside liner material and the backsheet material; and
a topical liner material coupled to the body facing surface of the bodyside liner material and having a depth dimension extending between a first body facing surface and a second, opposed garment facing surface, the topical liner material comprising a first zone and a second zone separated in the longitudinal direction, the first zone and the second zone each being individually contiguous and visually distinct,
wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material,
wherein the second zone comprises one or more protrusion and one or more aperture morphological features, and
wherein an average area of the aperture morphological features of the first zone are larger than an average area of the aperture morphological features of the second zone, and wherein:
the protrusion morphological features of the first zone have proximal ends and distal ends,
the embossment morphological features of the first zone have an opening comprising an opening width, a portion having an interior minimum width, and a portion having an interior maximum width with the portion having the interior minimum width disposed between, in a depth direction of the topical liner material, the opening and the portion having the interior maximum width, and
the protrusion morphological features are disposed within the embossment morphological features such that the proximal ends of the protrusion morphological features are disposed between, in a depth direction of the topical liner material, the portion of the embossment having the interior maximum width and the portion of the embossment having the interior minimum width.

2. The absorbent article of claim 1, wherein the morphological features of each of the first zone and the second zones define outer boundaries of the first zone and the second zone, the outer boundaries of the first zone forming a first shape and the outer boundaries of the second zone forming a second shape that is different from the first shape.

3. The absorbent article of claim 2, wherein the shapes defined by the outer boundaries of the first zone and the second zone comprise geometric shapes.

4. The absorbent article of claim 2, wherein the shape defined by the outer boundary of the second zone is a square and shape defined by the outer boundary of the first zone is a polygon have a number of sides greater than 4.

5. The absorbent article of claim 1, wherein a total area defined by the aperture morphological features of the first zone is greater than a total area defined by the aperture morphological features of the second zone.

6. The absorbent article of claim 1, wherein the protrusion morphological features of the second zone protrude for a distance away from the second garment facing surface that is less than a distance the protrusion morphological features of the first zone protrude away from the second garment facing surface.

7. The absorbent article of claim 1, wherein the second zone does not comprise embossment morphological features.

8. The absorbent article of claim 1, wherein each of the first zone and the second zone are disposed between 7.5 mm and 20 mm from longitudinally extending side edges of the topical liner material.

9. The absorbent article of claim 1, wherein the first zone is spaced longitudinally from the second zone between 5 mm and 30 mm.

10. The absorbent article of claim 1, wherein an area of the first zone is greater than an area of the second zone.

11. The absorbent article of claim 1, wherein an area of the first zone is between 4,000 mm and 8,000 mm and an area of the second zone is between 3,000 mm and 5,000 mm.

12. The absorbent article of claim 1, wherein a total area defined by the aperture morphological features of the first zone is between 2.5% and 14% of a total area of the first zone, and wherein a total area defined by aperture morphological features of the second zone is between 35% and 55% of a total area of the second zone.

13. The absorbent article of claim 1, wherein a total area defined by the embossment morphological features of the first zone is between 65% and 95% of a total area of the first zone.

14. The absorbent article of claim 1, wherein a total area defined by the aperture morphological features of the first zone is between 2.5% and 15% of a total area of the first zone.

15. The absorbent article of claim 1, wherein an average area defined by the aperture morphological features of the first zone is between 10% and 40% of an average area defined by the embossment morphological features of the first zone.

16. An absorbent article extending in a longitudinal direction and a transverse direction comprises:
a bodyside liner material having a depth dimension extending between a first body facing surface and a second, opposed garment facing surface, the bodyside liner material comprising a first zone and a second zone separated in the longitudinal direction, the first zone and the second zone each being individually contiguous and visually distinct;
a backsheet material; and
an absorbent body disposed between the bodyside liner material and the backsheet material;
wherein the first zone comprises one or more embossment, one or more aperture, and one or more protrusion morphological features with the one or more embossment morphological features extending from the first body facing surface toward the second garment facing surface and the one or more protrusion morphological features protruding from the second garment facing surface and toward the backsheet material,
wherein the second zone comprises one or more protrusion morphological features, and
wherein:
the protrusion morphological features of the first zone have proximal ends and distal ends,
the embossment morphological features of the first zone each have an opening comprising an opening width, interior portions defined by sidewalls of the embossment morphological features having an interior minimum width and an interior maximum width, with the interior minimum width disposed between the opening and the interior maximum width, and
the protrusion morphological features are disposed within the embossment morphological features such that the proximal ends of the protrusion morphological features are disposed between, in the depth direction of the bodyside liner material, the portions of the embossment morphological features which have the interior maximum width and the interior minimum width.

17. The absorbent article of claim 16, wherein a total area defined by the embossment morphological features of the first zone is between 65% and 95% of a total area of the first zone.

18. The absorbent article of claim 16, wherein a total area defined by the aperture morphological features of the first zone is between 2.5% and 14% of a total area of the first zone.

19. The absorbent article of claim 16, wherein an average area defined by the aperture morphological features of the first zone is between 10% and 40% of an average area defined by the embossment morphological features of the first zone.

20. The absorbent article of claim 1, wherein the distal ends of the protrusion morphological features define the aperture morphological features, and wherein the protrusion morphological features are funnel shaped such that openings of the protrusion morphological features defined by the proximal ends of the protrusion morphological features have greater areas than the aperture morphological features defined by the distal ends of the protrusion morphological features.
